(19) United States
(12) Patent Application Publication
(10) Pub. No.: US 2022/0338769 A1
(43) Pub. Date: Oct. 27, 2022

(54) METHODS AND APPARATUS FOR SUBSTANCE DELIVERY IN AN IMPLANTABLE DEVICE

(71) Applicant: GlySens Incorporated, San Diego, CA (US)

(72) Inventors: Lev Kurbanyan, San Diego, CA (US); Jean Sebastien Pradel, Chula Vista, CA (US); Timothy Routh, San Diego, CA (US); Brad Walker, San Diego, CA (US); Ted Williams, Carlsbad, CA (US)

(21) Appl. No.: 17/690,689

(22) Filed: Mar. 9, 2022

Related U.S. Application Data
(60) Provisional application No. 63/179,910, filed on Apr. 26, 2021.

(51) Int. Cl. A61B 5/1486; A61B 5/145; A61B 5/07; A61K 31/573; A61K 47/34

(52) U.S. Cl. CPC A61B 5/14865; A61B 5/14532; A61B 5/076; A61K 31/573; A61K 47/34

(57) ABSTRACT

Apparatus and methods for mitigation and control of inflammatory responses during usage of an implantable sensor device. In one exemplary embodiment, the implantable sensor device includes a drug-eluting component configured to release a varying amount of anti-inflammatory substance(s) (such as corticosteroid) over the lifetime of the implant, such as according to a desired elution profile. In one variant, this component is also an analyte-permeable membrane used as part of a detector of the implant. Inhibition of inflammation in the tissue improves availability of analytes (such as oxygen and glucose) to the sensor in addition to reducing fibrous encapsulation. Various modifications to the elution rate, and configuration of the implant, allow optimized control over undesirable effects such as foreign body reactions (FBR) or other inflammatory responses which may reduce usable implant lifetime.

### PRIORITY AND RELATED APPLICATIONS

This application claims priority to and benefit of co-pending U.S. Provisional Patent Application Ser. No. 63/179,910 filed Apr. 26, 2021 and entitled “METHODS AND APPARATUS FOR SUBSTANCE DELIVERY IN AN IMPLANTABLE DEVICE”, which is incorporated by reference herein in its entirety.

This application is generally related to potions of the subject matter contained in co-owned and/or co-pending U.S. patent application Ser. No. 13/559,475 filed Jul. 26, 2012 entitled “Tissue Implantable Sensor With Hermetically Sealed Housing,” issued as U.S. Pat. No. 10,561,351 on Feb. 18, 2020, Ser. No. 14/982,346 filed Dec. 29, 2015 and entitled “Implantable Sensor Apparatus and Methods”, issued as U.S. Pat. No. 10,660,550 on May 26, 2020, Ser. No. 15/170,571 filed Jun. 1, 2016 and entitled “Biocompatible Implantable Sensor Apparatus and Methods”, issued as U.S. Pat. No. 10,561,353 on Feb. 18, 2020, Ser. No. 15/197,104 filed Jun. 29, 2016 and entitled “Bio-adaptable Implantable Sensor Apparatus and Methods”, issued as U.S. Pat. No. 10,638,962 on May 5, 2020, Ser. No. 15/359,406 filed Nov. 22, 2016 and entitled “Heterogeneous Analyte Sensor Apparatus and Methods”, Ser. No. 15/368,436 filed Dec. 2, 2016 and entitled “Analyte Sensor Receiver Apparatus and Methods”, and Ser. No. 15/472,091 filed Mar. 28, 2017 and entitled “Analyte Sensor User Interface Apparatus and Methods,” each of the foregoing incorporated herein by reference in its entirety.

This application is also generally related to portions of the subject matter contained in co-owned and/or co-pending U.S. patent application Ser. No. 15/645,913 filed Jul. 10, 2017 entitled “Analyte Sensor Data Evaluation and Error Reduction Apparatus and Methods,” issued as U.S. Pat. No. 10,638,979 on May 5, 2020, and U.S. patent application Ser. No. 16/233,536 filed Dec. 27, 2018 entitled “Apparatus and Methods for Analyte Sensor Mismatch Correction,” each of the foregoing incorporated herein by reference in its entirety.

This application is also generally related to portions of the subject matter contained in co-owned and/or co-pending U.S. patent application Ser. No. 16/443,684 filed Jun. 17, 2019 and entitled “Analyte Sensor Apparatus and Methods,” which claims the benefit of priority to co-owned and co-pending U.S. Provisional Patent Application No. 62/687,115 filed on Jun. 19, 2018 and entitled “Analyte Sensor Apparatus and Methods,” as well as co-owned and co-pending U.S. patent application Ser. No. 16/453,794 filed Jun. 26, 2019 and entitled “Apparatus and Methods for Analyte Sensor Spatial Mismatch Mitigation and Correction,” which claims the benefit of priority to co-owned and co-pending U.S. Provisional Patent Application No. 62/690,745 filed on Jun. 27, 2018 and entitled “Apparatus and Methods for Analyte Sensor Spatial Mismatch Correction,” each of the foregoing incorporated herein by reference in its entirety.

This application is also generally related to portions of the subject matter contained in co-owned and/or co-pending U.S. patent application Ser. No. 16/882,182 filed May 22, 2020 and entitled “Implantable Sensor Apparatus and Methods,” U.S. patent application Ser. No. 16/866,424 filed May 4, 2020 and entitled “Bio-Adaptable Implantable Sensor Apparatus and Methods,” U.S. patent application Ser. No. 16/989,721 filed Aug. 10, 2020 and entitled “Bio-Adaptable Implantable Sensor Apparatus and Methods,” U.S. patent application Ser. No. 16/866,436 filed May 4, 2020 and entitled “Analyte Sensor Data Evaluation and Error Reduction Apparatus and Methods,” U.S. patent application Ser. No. 16/253,095 filed Jan. 21, 2019 and entitled “Biocompatible Implantable Sensor Apparatus and Methods,” U.S. patent application Ser. No. 15/853,574 filed Dec. 22, 2017 and entitled “Analyte Sensor and Medicant Delivery Data Evaluation and Error Reduction Apparatus and Methods,” U.S. patent application Ser. No. 16/055,547 filed Aug. 6, 2018 and entitled “Hermetic Implantable Sensor,” U.S. patent application Ser. No. 16/601,346 filed Oct. 14, 2019 and entitled “Tissue Implantable Sensor with Hermetically Sealed Housing,” U.S. patent application Ser. No. 16/256,939 filed Jan. 24, 2019 and entitled “Tissue Implantable Sensor with Hermetically Sealed Housing,” U.S. Provisional Patent Application Ser. No. 63/134,869 filed Jan. 7, 2021 and entitled “Methods and Apparatus for Error Mitigation and Difference Determination,” U.S. patent application Ser. No. 15/919,052 filed Mar. 12, 2018 and entitled “Method of Manufacturing an Analyte Detector Element,” issued as U.S. Pat. No. 10,736,553 on Aug. 11, 2020, U.S. patent application Ser. No. 13/024,209 filed Feb. 9, 2011 and entitled “Hermetic Implantable Sensor,” issued as U.S. Pat. No. 10,041,897 on Aug. 7, 2018, U.S. patent application Ser. No. 11/479,331 filed Jun. 30, 2006 and entitled “Hermetic Feedthrough Assembly for Ceramic Body,” issued as U.S. Pat. No. 8,763,246 on Jul. 1, 2014, and U.S. patent application Ser. No. 14/314,596 filed Jun. 25, 2014 and entitled “Hermetic Feedthrough Assembly for Ceramic Body,” issued as U.S. Pat. No. 9,782,111 on Oct. 10, 2017, each of the foregoing incorporated herein by reference in its entirety.

### COPYRIGHT

A portion of the disclosure of this patent document contains material that is subject to copyright protection. The copyright owner has no objection to the facsimile reproduction by anyone of the patent document or the patent disclosure, as it appears in the Patent and Trademark Office patent files or records, but otherwise reserves all copyright rights whatsoever.

### 1. TECHNICAL FIELD

The disclosure relates generally to the field of implantable sensors. In one specific aspect, the disclosure relates to enhanced delivery of blood analytes and prevention of excessive fibrosis surrounding an implanted sensor, via controlled drug elution and/or device modification.

### 2. DESCRIPTION OF RELATED TECHNOLOGY

Implantable electronics is a rapidly expanding discipline within the medical arts. Owing in part to significant advances in electronics and wireless technology integration, miniaturization, performance, and material biocompatibility, sensors or other types of electronics which once were beyond the realm of reasonable use within a living subject (i.e., in vivo) can now be surgically implanted within such subjects with minimal effect on the recipient subject, and in fact convey many inherent benefits.

One particular area of note relates to blood analyte monitoring for subjects, such as for example glucose monitoring for those with “type 1” or “type 2” diabetes. As is well known, regulation of blood glucose is impaired in people with diabetes by: (1) the inability of the pancreas to adequately produce the glucose-regulating hormone insulin; (2) the insensitivity of various tissues that use insulin to take up glucose; or (3) a combination of both of these phenomena. Safe and effective correction of this dysregulation requires blood glucose monitoring.

Implantable glucose sensors (e.g., continuous glucose monitoring sensors) have long been considered as an alternative to intermittent monitoring of blood glucose levels by the “fingersticking” method of sample collection (which has several disadvantages, including discomfort, insufficient sampling, and user involvement). These devices may be fully implanted, where all components of the system reside within the body and there are no through-the-skin (i.e., percutaneous) elements, or they may be partially implanted, where certain components reside within the body but are physically connected to additional components external to the body via one or more percutaneous elements. Further, such devices (especially fully implantable devices) provide users a great deal of freedom from potentially painful (and not always optimally timed) intermittent sampling methods such as fingersticking, as well as having to remember and obtain self-administered blood analyte readings.

In conventional sensors, accuracy can be adversely affected by a myriad of factors, one of which is the body's natural defenses to foreign object (e.g., an implanted sensor), resulting in tissue response (e.g., encapsulation of an implant, inflammation, protein adsorption on the foreign body). Such foreign body response (FBR) or other inflammatory tissue responses can directly and indirectly affect the accuracy and precision of analyte measurements by interfering with glucose measurements as well as measurements indicative of or related to the presence of glucose, including (as one example) determination of blood oxygen concentration based on measurements of partial pressure of oxygen (pO2). In other words, excessive fibrous encapsulation poses a problem for usage of the implanted device as it limits analytes of interest (e.g., oxygen and glucose) from reaching the sensor in sufficient quantity due to, among other things, poor blood flow/vasularization of the fibrous encapsulation proximate to the sensor detecting element(s). As such, there is a need for control or mitigation of FBR and other undesirable tissue responses to implantable devices.

Some solutions have been developed in the industry to attempt to address these issues. Such solutions generally fall into two categories: (1) controlled elution of anti-inflammatory substances from the device (e.g., anti-inflammatory drugs and anti-fibrotic agents), and (2) device surface modification (e.g., hydrogel or polymer coatings) to prevent macrophage adhesions and thus excessive encapsulation.

However, it has been unclear to what degree, where within the tissue, and how such substances should be introduced and eluted over time, particularly over the long term. That is, current solutions are not yet ideal to combat the inflammatory response to implantation of sensors or other implantable devices, especially with regard to particular analytes.

Moreover, such extant solutions are not targeted or related to a particular analyte, but rather more generally to reduce inflammation.

Thus, improved apparatus and methods addressing the foregoing needs, including inter alia, optimized control of drug elution so as to provide improvement of sensor and blood analyte measurement/calibration accuracy, as well as reduction of measurement errors, are needed.

### SUMMARY

The present disclosure satisfies the foregoing needs by providing, inter alia, improved apparatus (including an implanted sensor and associated logic) and methods, for enhanced control of substance storage and elution from an implanted device to mitigate inflammatory bodily responses thereto, including with respect to particular analytes.

In one aspect of the present disclosure, a method for mitigating inflammatory bodily response to an implantable device is disclosed. In one embodiment, the method includes identifying at least one anti-inflammatory substance for use with the implantable device; determining a desired elution profile for the at least one anti-inflammatory substance; obtaining a substance-eluting component according to the determined elution profile; and incorporating the substance-eluting component with the implantable device.

In another aspect of the present disclosure, an implantable apparatus capable of mitigating inflammatory bodily responses is disclosed.

In a further aspect, a method of extending the implantation longevity of an implantable medical device such as analyte sensor or medicant delivery device is disclosed.

In a further aspect, a methods and apparatus for mitigating effects on analyte concentration are disclosed. In one embodiment, the methods and apparatus include impregnating or otherwise including an elutable substance within a membrane which is also configured to pass analyte molecules from a user's tissue to one or more analyte sensors.

In a further aspect, methods and apparatus for controlling the long-term elution of one or more target substances are disclosed. In one embodiment, the target substance is comprised of one or more corticosteroids such as e.g., Dexamethasone (including one or more of the various species thereof), and its elution is controlled over a period of multiple months.

In another aspect, an elution component for use in an implantable sensing or other apparatus is disclosed. In one embodiment, the elution component includes some level of porosity such that one or more target substances such as anti-inflammatory agent(s) may be eluted from the component over time. In one variant, the component is a separate component which has no other function than elution and inflammation control. In another variant, the component is integrated with or is another active or passive component of the host platform (e.g., an analyte membrane through which e.g., oxygen or glucose molecules may pass, or a non-permeable enzymatic matrix retaining membrane of a sensor).

In one implementation, the elution component is formed as a unitary component that is substantially planar. In another implementation, the components has a geometric cross-sectional shape, such as circular, oval, square, trapezoidal, etc. In a further implementation, the component is formed as two or more discrete distributed components, such as across or proximate to an active region of an implantable sensor face.

In another embodiment, the component includes one or a plurality of target substances (e.g., one or more corticosteroids) which are impregnated into the component. In one such approach, the target substance(s) is/are formed as a powder which is intermixed within the matrix of the component (e.g., silicone rubber prior to being cured). In other approaches, some or all of the target substance(s) are disposed within the component(s) after the latter's formation.

In some embodiments, mixtures of target substances (such as two species of Dexamethasone, or Dexamethasone and another steroid) are used in order to tailor the elution and anti-inflammatory profiles applied to the tissue during implantation. In some variants, solubility of the target substance is a factor considered in controlling the elution profile.

In a further aspect, method and apparatus for controlling the systemic amount of target substance(s) delivered to the user over time are disclosed.

In another aspect, method and apparatus for controlling inflammatory response over multiple (e.g., two or more) successive implantations are disclosed. In one embodiment, the methods and apparatus make use of differing target substances, target substance loadings, and/or elution profiles so as to leverage differential response by the user's body over the successive implantations.

According to some methods and apparatus described herein, a desired eluting rate and profile is selected specifically for usage with the drug-eluting component. The eluting component is placed onto the implant, and the implant is placed into the host tissue.

Other features and advantages of the present disclosure will immediately be recognized by persons of ordinary skill in the art with reference to the attached drawings and detailed description of exemplary embodiments as given below.

### BRIEF DESCRIPTION OF THE DRAWINGS

FIG. 1A is a graphical diagram comparing oxygen levels measured by sensor electrodes without dexamethasone-eluting components and those with dexamethasone-eluting components.

FIG. 1B is a graphical diagram comparing fibrous encapsulation thicknesses over the sensing region of sensors measured using fixed tissue slices from sensors without dexamethasone-eluting components and those with dexamethasone-eluting components.

FIG. 1C is a graphical diagram comparing fibrous encapsulation thickness differences between the sensing and non-sensing regions of sensors without dexamethasone-eluting components and those with dexamethasone-eluting components.

FIG. 1D is a graphical diagram comparing oxygen levels measured by sensor electrodes without dexamethasone-eluting components and those with dexamethasone-eluting components.

FIG. 1E is a graphical diagram comparing oxygen levels measured by sensor electrodes with dexamethasone-eluting components, implanted on muscle, fascia, and subcutaneous fat.

FIG. 2 is a graph illustrating total release of dexamethasone acetate (DXA) from an implantable sensor device and daily release of DXA from the implantable sensor over time.

FIGS. 3A-3C are top perspective, side and front views respectively of one exemplary embodiment of a fully implantable biocompatible sensor apparatus useful with various aspects of the present disclosure.

FIGS. 3D-3E are bottom and top elevation views respectively of one exemplary embodiment of a fully implantable biocompatible sensor apparatus useful with various aspects of the present disclosure.

FIGS. 3F-3H are top and bottom perspective transparent views of the sensor apparatus of FIGS. 1A-1E, showing various internal components and layout.

FIG. 4 is a top elevation view of an exemplary configuration of a drug-eluting component incorporated around a sensor area of the implant device of FIGS. 3A-3H.

FIG. 5 is a side cross-sectional view of one exemplary detector element (i.e., enzyme matric-based) of a detector array in a fully implantable sensor apparatus according to one embodiment of the present disclosure.

FIG. 5A is a side cross-sectional view of one exemplary spout region (outer non-enzyme membrane removed) of a detector element of a detector array in a fully implantable sensor apparatus according to one embodiment of the present disclosure.

FIG. 5B is a top elevation view of one exemplary embodiment of an enzymatic detector element with membrane shell and membrane configured for substance delivery according to the present disclosure.

FIG. 6A is a top plan view of another exemplary implantable sensor apparatus with substance delivery, according to one embodiment of the present disclosure.

FIG. 6B is a top plan view of an exemplary configuration of a sensor face of the implantable sensor apparatus of FIG. 6A, according to one embodiment of the present disclosure.

FIG. 6C is a top plan view of an exemplary configuration of a sensor element group (including multiple reference electrodes associated with a single working electrode) of the sensor face of FIG. 6B, according to one embodiment of the present disclosure.

FIG. 7 is a logical flow diagram of an exemplary embodiment of a generalized method for mitigating anti-inflammatory bodily reactions caused by an implantable device.

FIG. 8 is a logical flow diagram of an exemplary embodiment of a generalized method for fabricating an implantable device configured for substance delivery according to the present disclosure.

FIG. 9 is a logical flow diagram of an exemplary embodiment of a method for mitigating anti-inflammatory bodily reactions caused by an implantable device during operation thereof.

FIG. 10 is a logical flow diagram of an exemplary embodiment of a method for configuring an implant utilizing drug eluting capability based on implantation history.

All Figures © Copyright 2018-2021 GlySens Incorporated. All rights reserved.

### DETAILED DESCRIPTION

Reference is now made to the drawings, wherein like numerals refer to like parts throughout.

### Overview

In exemplary aspects, the present disclosure provides method and apparatus which enable control and management of inflammatory responses to implants in bodily tissue so as to, among other things, enhance implant performance and longevity.

In one exemplary embodiment, one or more drug-eluting components is/are adhered to a surface around a sensor area of an implantable device, such as a blood analyte sensor device. In other variants, the elution functionality is integrated within one or more components of the sensor device, such as an analyte-permeable membrane thereof. The sensor area of the implant is configured to detect analytes of interest, such as oxygen (partial pressure of oxygen, pO2) or glucose, while the device is implanted within a host body. The drug-eluting component is in one implementation formed by e.g., immobilizing dexamethasone acetate (DXA) within a matrix or substrate such as a sheet of implant-grade silicone rubber (SR). Other forms of dexamethasone (e.g., dexamethasone sodium phosphate (DSP) may be used alternately to or together with DXA to modify, e.g., elution profiles, according to the prescribed application.

Dexamethasone is a corticosteroid that inhibits inflammation, thus reducing fibrous encapsulation. Further, the Assignee hereof has recognized that use of dexamethasone and/or other such substances also improves the availability of analytes or species of interest (e.g., glucose or oxygen) to the sensor. In order for the dexamethasone to be optimally effective for long term sensor operation, a small amount of the drug is, in some disclosed embodiments, slowly eluted over the life of the implant according to a non-linear profile. The acetate salt of dexamethasone has low water solubility, which slows the release of dexamethasone when impregnated into a matrix material. Therefore, it is advantageous to elute dexamethasone acetate (DXA) into the implanted area in a controlled manner, to the degree desired and necessary.

The blood analyte sensor in some embodiments disclosed herein and used with the exemplary drug-eluting component is a blood glucose monitor utilizing oxygen-based sensing, with the blood analyte detectors include oxygen and glucose electrodes (e.g., arranged as differential sensor pairs as in the exemplary analyte sensor manufactured by the Assignee hereof and described in co-owned U.S. patent application Ser. Nos. 13/559,475, 14/982,346, 15/170,571, 15/197,104, 15/359,406, 15/645,913, and 16/233,536 each previously incorporated herein, or arranged as differential sensor groups as in the exemplary “GEN 3” device also manufactured by the Assignee hereof and described in co-owned U.S. patent application Ser. Nos. 16/443,684 and 16/453,794, each previously incorporated herein.

### Detailed Description of Exemplary Embodiments

Exemplary embodiments of the present disclosure are now described in detail. While these embodiments are primarily discussed in the context of a fully implantable “GEN 3” glucose sensor and associated methods such as described in U.S. patent application Ser. Nos. 16/443,684 and 16/453,794, it will be recognized by those of ordinary skill that the present disclosure is not so limited. For instance, the configurations described in U.S. Patent Application Publication No. 2013/0197332 filed Jul. 26, 2012 entitled “Tissue Implantable Sensor With Hermetically Sealed Housing”; U.S. Pat. No. 7,894,870 to Lucisano et al. issued Feb. 22, 2011 and entitled “Hermetic Implantable Sensor”; U.S. Patent Application Publication No. 2011/0137142 to Lucisano et al. published Jun. 9, 2011 and entitled “Hermetic Implantable Sensor”; U.S. Pat. No. 8,763,245 to Lucisano et al. issued Jul. 1, 2014 and entitled “Hermetic Feedthrough Assembly for Ceramic Body”; U.S. Patent Application Publication No. 2014/0309510 to Lucisano et al. published Oct. 16, 2014 and entitled “Hermetic Feedthrough Assembly for Ceramic Body”; U.S. Pat. No. 7,248,912 to Gough et al. issued Jul. 24, 2007 and entitled “Tissue Implantable Sensors for Measurement of Blood Solutes”; and U.S. Pat. No. 7,871,456 to Gough et al. issued Jan. 18, 2011 and entitled “Membranes with Controlled Permeability to Polar and Apolar Molecules in Solution and Methods of Making Same”; U.S. Patent Application Publication No. 2013/0197332 to Lucisano et al. published Aug. 1, 2013 and entitled “Tissue Implantable Sensor with Hermetically Sealed Housing”; and PCT Patent Application Publication No. 2013/016573 to Lucisano et al. published Jan. 31, 2013 and entitled “Tissue Implantable Sensor with Hermetically Sealed Housing,” each of the foregoing incorporated herein by reference in its entirety, as well as those of U.S. patent application Ser. Nos. 13/559,475; 14/982,346; 15/170,571; 15/197,104; 15/359,406; 15/368,436; 15/472,091; 15/645,913; 15/853,574; and Ser. No. 16/233,536, each previously incorporated herein, may each individually or in combinations thereof be used as the basis for the improved apparatus and methods described herein.

Further, the various aspects of the disclosure are useful with, inter alia, other types of implantable sensors, implantable pumps, and/or other implantable devices which may benefit from controlled inflammatory response.

Further, while the following embodiments describe specific implementations of e.g., biocompatible oxygen-based multi-sensor element devices for measurement of glucose having specific configurations, protocols, locations, and orientations for implantation (e.g., sensor implantation proximate the waistline on a human abdomen with the sensor array disposed proximate to fascial tissue; see e.g., U.S. patent application Ser. No. 14/982,346, entitled “Implantable Sensor Apparatus and Methods” and filed Dec. 29, 2015, now U.S. Pat. No. 10,660,550, previously incorporated herein), those of ordinary skill in the related arts will readily appreciate that such descriptions are purely illustrative, and in fact the methods and apparatus described herein can be used consistent with, and without limitation: (i) in living beings other than humans; (ii) other types or configurations of sensors (e.g., other types, enzymes or analytes, and/or theories of operation of glucose sensors, sensors other than glucose sensors, such as e.g., sensors for other analytes such as urea, lactate); (iii) other implantation locations and/or techniques (including without limitation transcutaneous or non-implanted devices as applicable); and/or (iv) other devices (e.g., other sensor apparatus, medicant delivery devices, non-sensor devices, and non-substance delivery devices).

As used herein, the term “analyte” refers without limitation to a substance or chemical species that is of interest in an analytical procedure. In general, the analyte itself may or may not be directly measurable, in cases where it is not, a measurement of the analyte (e.g., glucose) can be derived through measurement of chemical constituents, components, or reaction byproducts associated with the analyte (e.g., hydrogen peroxide, oxygen, free electrons, etc.).

As used herein, the terms “delivery device” and “medicant delivery device” refer to a device configured for delivery of solutes, including without limitation one or more mechanical or electro-mechanical pumps, such as partially implanted or fully implanted pumps, as well as other delivery modes such as diffusion (e.g., through a membrane or other barrier), or even dissolution of solids. Exemplary partially implantable pumps include transcutaneous pumps which include an implantable portion (e.g., a cannula, a needle, etc.) coupled to a non-implantable portion (e.g., a housing, a reservoir, a pump actuator, etc.). Exemplary fully implantable pumps include subcutaneous pumps, which are implanted beneath the skin of a user and are in data communication with an external controlling (e.g., processing) apparatus.

As used herein, the terms “detector” and “sensor” refer without limitation to a device having one or more elements (e.g., detector element, sensor element, sensing elements, etc.) that generate, or can be made to generate, a signal indicative of a measured parameter, such as the concentration of an analyte (e.g., glucose) or its associated chemical constituents and/or byproducts (e.g., hydrogen peroxide, oxygen, free electrons, etc.). Such a device may be based on electrochemical, electrical, optical, mechanical, thermal, or other principles as generally known in the art. Such a device may consist of one or more components, including for example, one, two, three, or four electrodes, and may further incorporate immobilized enzymes or other biological or physical components, such as membranes, to provide or enhance sensitivity or specificity for the analyte.

As used herein, the terms “orient,” “orientation,” and “position” refer, without limitation, to any spatial disposition of a device and/or any of its components relative to another object or being, and in no way connote an absolute frame of reference.

As used herein, the terms “top,” “bottom,” “side,” “up,” “down,” and the like merely connote, without limitation, a relative position or geometry of one component to another, and in no way connote an absolute frame of reference or any required orientation. For example, a “top” portion of a component may actually reside below a “bottom” portion when the component is mounted to another device (e.g., host sensor).

As used herein, the term “application” (or “app”) refers generally and without limitation to a unit of executable software that implements a certain functionality or theme. The themes of applications vary broadly across any number of disciplines and functions (such as on-demand content management, e-commerce transactions, brokerage transactions, home entertainment, calculator etc.), and one application may have more than one theme. The unit of executable software generally runs in a predetermined environment; for example, the Java® environment.

As used herein, the term “computer program” or “software” is meant to include any sequence or human or machine cognizable steps which perform a function. Such program may be rendered in virtually any programming language or environment including, for example, C/C++, Fortran, COBOL, PASCAL, assembly language, markup languages (e.g., HTML, SGML, XML, VoXML), and the like, as well as object-oriented environments such as the Common Object Request Broker Architecture (CORBA), Java® (including J2ME, Java Beans, etc.) and the like. Applications as used herein may also include so-called “containerized” applications and their execution and management environments such as VMs (virtual machines) and Docker and Kubernetes.

As used herein, the terms “Internet” and “internet” are used interchangeably to refer to inter-networks including, without limitation, the Internet. Other common examples include but are not limited to: a network of external servers, “cloud” entities (such as memory or storage not local to a device, storage generally accessible at any time via a network connection, or cloud-based or distributed processing or other services), service nodes, access points, controller devices, client devices, etc.

As used herein, the term “long term” refers without limitation to periods of time which may be measured in e.g., whole months or years.

As used herein, the term “memory” includes any type of integrated circuit or other storage device adapted for storing digital data including, without limitation, ROM, PROM, EEPROM, DRAM, SDRAM, DDR/2 SDRAM, EDO/FPMS, RLDRAM, SRAM, “flash” memory (e.g., NAND/NOR), 3D memory, HBM/HBM2, and PSRAM.

As used herein, the terms “microprocessor” and “processor” or “digital processor” are meant generally to include all types of digital processing devices including, without limitation, digital signal processors (DSPs), reduced instruction set computers (RISC), general-purpose (CISC) processors, SoCs, microprocessors, gate arrays (e.g., FPGAs), PLDs, state machines, reconfigurable computer fabrics (RCFs), array processors, secure microprocessors, and application-specific integrated circuits (ASICs). Such digital processors may be contained on a single unitary integrated circuit (IC) die, or distributed across multiple components.

As used herein, the term “interface” refers to any signal or data interface with a component or network including, without limitation, those of the FireWire (e.g., FW400, FW800, etc.), USB (e.g., USB 2.0, 3.0. OTG), Ethernet (e.g., 10/100, 10/100/1000 (Gigabit Ethernet), 10-Gig-E, etc.), MoCA, LTE/LTE-A, 5G NR, Wi-Fi (802.11), WiMAX (802.16), Z-wave, PAN (e.g., 802.15)/Zigbee, 5G NR (3GPP), Bluetooth, Bluetooth Low Energy (BLE) or power line carrier (PLC) families.

As used herein, the term “wireless” means any wireless signal, data, communication, or other interface including without limitation Wi-Fi, Bluetooth (including BLE or “Bluetooth Smart”), NFC, 3G (3GPP/3GPP2), HSDPA/HSUPA, TDMA, CDMA (e.g., IS-95A, WCDMA, etc.), FHSS, DSSS, GSM, PAN/802.15, WiMAX (802.16), 802.20, Zigbee®, Z-wave, narrowband/FDMA, OFDM, PCS/DCS, LTE/LTE-A/LTE-U/LTE-LAA, 5G-NR (3GPP), analog cellular, CDPD, satellite systems, millimeter wave or microwave systems, acoustic, and infrared (i.e., IrDA).

### Control of Fibrous Encapsulation

As referenced above, the compatibility of an implanted device with bodily tissue is of paramount importance for proper operation of the implant. Excessive fibrous encapsulation (caused by inflammatory tissue responses, e.g., foreign body reactions (FBR)) reduces the efficacy of such an implanted device. For proper operation, implanted devices generally require substances or analytes of interest to reach the sensor(s) disposed on or in the implanted device. Hence, encapsulation of the implant tends to prevent analytes of interest from reaching the sensors on/in the device. In the context of the present discussion, oxygen-based glucose sensors may be configured to measure and determine, inter alia, oxygen concentrations for purposes of monitoring blood glucose levels of a patient. “Walling off” portions of the implanted device may impede not only detection and measurement of analytes of interest such as glucose and oxygen within desired levels of accuracy, but may also reduce the usable (implanted) lifetime of the device.

As background, solutions such as eluting drugs (e.g., steroids) to locally suppress inflammation and modifying device surfaces exist. Prior experiments with various types of predicate devices have been performed regarding drug elution. Specifically, in these experiments, release of dexamethasone from an implanted sensor device was studied, particularly dexamethasone acetate (DXA). Dexamethasone is a synthetic corticosteroid and has been used to inhibit inflammation.

As described in detail herein, the present disclosure leverages the desirable properties of anti-inflammatory agents such as DXA for reducing fibrous encapsulation and FBR, and notably improve the availability of glucose-modulated oxygen and other species to the sensor detector elements during implanted operation.

As a brief aside, dexamethasone as a potent corticosteroid may be administered acutely and chronically in a variety of routes for the purposes of treatment and diagnosis. Therapeutic dosages usually range from 4 to 20 mg per day for adults, and 1 mg is typically given for dexamethasone suppression tests. As a synthetic compound, dexamethasone is absent in normal serum. Serum levels of approximately 130-200 ng/dL and greater are correlated with cortisol suppression.

Other compounds of dexamethasone exist, such as dexamethasone sodium phosphate (DSP), which is particularly suitable for, e.g., intravenous administration because it is highly water soluble; relatively large doses may be diluted and administered in a small volume.

Notably, the acetate salt of dexamethasone has low water solubility, which slows the dissolution and release of dexamethasone when delivered by a component such as when impregnated into a matrix of a material such as silicone rubber or other elastomer/polymer. Further, as will be discussed further below, usage of combinations of different amounts of dexamethasone components (e.g., DXA and DSP) may achieve desired elution profiles or other properties that each one alone cannot provide.

Hence, as a foundation for development of the exemplary methods and apparatus described herein, the following sample case studies summarize experimental results using predicate devices, i.e., medical devices legally marketed within the United States and used as a point of comparison for new medical devices. The predicate devices were investigated to guide the release profile target for the exemplary sensor and component disclosed herein.

### Case Study #1

In one experiment, a subcutaneously implanted glucose sensor approved for up to 90 days of use (within the United States) was studied. This device has a silicone collar containing 1.75 mg DXA, of which 180-350 μg is reported to be released over 90 days, for an average release of 2-3.9m/day. When one such device was implanted, there was no measurable DXA reported in patients' plasma, given a limit of quantification (LOQ, the lowest concentration of the analyte that can be reliably quantified)=50 pg/mL. When two devices were implanted to double DXA exposure, DXA was measurable in some patients for up to 8 days after implant. The maximum reported plasma DXA was 114 pg/mL. Based on an analysis of the reported drug release, it is likely that a daily dose of approximately 15 μg DXA or higher is required to have a DXA level greater than the LOQ in the patient's plasma.

### Case Study #2

Leads containing a maximum of 1 mg dexamethasone sodium phosphate (DXSP) have been on the market since the 1980s. The DXSP is intended to prevent fibrous encapsulation of the lead. One retrospective study evaluated leads that had been removed from human and canine subjects for residual DXSP. See Mond HG, Stokes KB. The steroid-eluting electrode: a 10-year experience. Pacing Clin Electrophysiol. 1996; 19(7):1016-1020. doi:10.1111/j.1540-8159.1996.tb03407.x, incorporated herein by reference in its entirety.

The results indicated that after 1 year, between 30 and 40% of the DXSP remains. This corresponds to a release of 0.6-0.7 mg in the first year, or an average of 1.6-1.9 μg/day.

### Case Studies #3 and #4

The studies summarized above indicate that different daily amounts of dexamethasone may be released. Further, the Assignee hereof has recognized that location of the dexamethasone-eluting component may affect oxygen signals detected by the sensor, and that further optimizations are possible with respect to prevention of encapsulation and mitigation of the adverse effects of inflammatory responses on the functionalities of the implanted device (e.g., proper measurements of analytes by the implanted device and its sensor(s)).

To that end, the Assignee hereof has performed tests (including pre-clinical animal studies) and obtained experimental data on both of the aforementioned general solutions (drug elution and device modification) to modulate the effects of FBR. Particularly, in vivo studies relating to elution of anti-inflammatory substances and implant locations have been performed, with the findings thereof summarized below.

Case Study #3. Evaluation of Sensors' Tissue Response and the Effectiveness of any Design Changes for Improving Response in a Chronic Porcine Model—

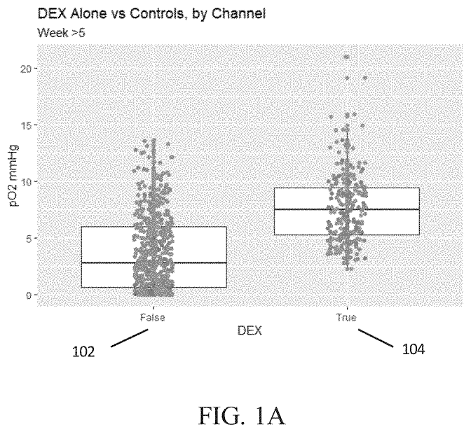

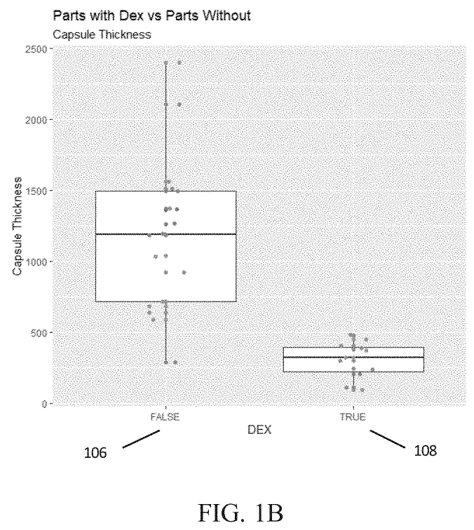

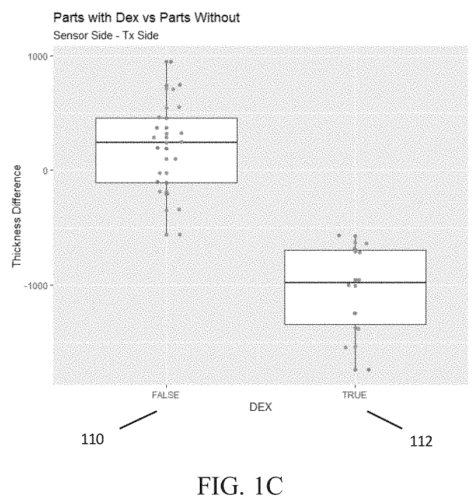

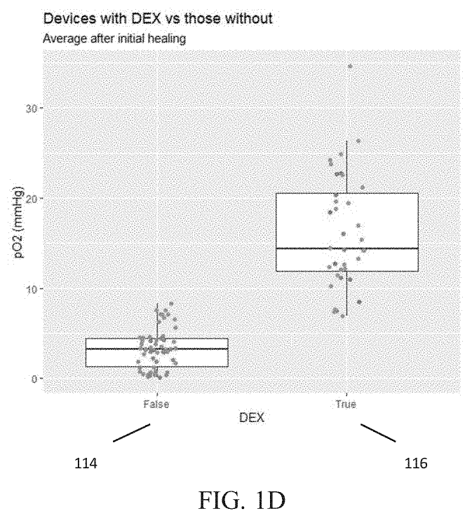

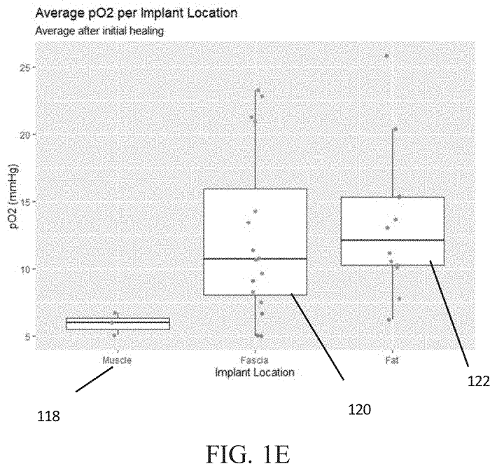

In one test, a total of 28 sensors of a first type with various design attributes were implanted in a total of eight Yucatan pigs to evaluate feasibility of augmenting the oxygen delivery to the sensor over a four-month period. Sensor signals and tissue histology were utilized for evaluation. One sensor of a second type was implanted in a pig as a proof-of-concept of Bluetooth Low Energy (BLE) communication of the type described previously herein with respect to the exemplary embodiments of FIGS. 1A-1J.

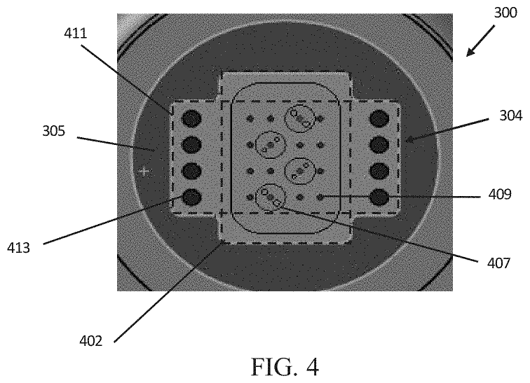

Findings included:
1. Sensors with dexamethasone-eluting components had an overall higher oxygen signal at the sensor array than those without dexamethasone. FIG. 4A herein illustrates oxygen levels measured by sensor electrodes without dexamethasone-eluting components 402 and those with dexamethasone-eluting components 404.
2. The fibrous capsule in the region over the sensor array had a lower overall thickness in sensors with dexamethasone eluting components than those without. FIG. 4B illustrates possible fibrous capsule thicknesses over the sensing region of sensors measured using fixed tissue slices from sensors without dexamethasone-eluting components 406 and those with dexamethasone-eluting components 408. FIG. 4C illustrates fibrous capsule thickness differences between the sensing and non-sensing regions of sensors without dexamethasone-eluting components 410 and those with dexamethasone-eluting components 412.
3. The fibrous capsule, in the region over the sensor array, had a lower overall thickness that the capsule on the same sensor in a control region for those sensors containing a Dexamethasone eluting component.
4. The sensor of the second type exhibited adequate Bluetooth Low Energy communication to support experiment data transfer.

In this study, the dexamethasone-eluting component advantageously reduced the capsule thickness in a localized region of the sensor face and increased oxygen delivery to the sensor array.

Case Study #4. Evaluation of the Performance of the Sponsor's Blood Glucose Monitoring Devices with Monthly Excursions in Chronic Yucatan Mini-Pig Model—

In another test, a total of 16 sensors of a first type and a total of 12 sensors of a second type were implanted in a total of six Yucatan pigs for a four-month duration to evaluate: (1) feasibility of augmenting the oxygen delivery to the sensor using a refined dexamethasone-eluting component, (2) feasibility of oxygen delivery to the sensor of the second type at various implant depths. Sensor signals were utilized for evaluation. Specifically, the shape of the exemplary component was adjusted such that it was closer and more contoured to the sensing array (i.e., generally of the configuration shown in FIG. 4 discussed subsequently herein), whereas in case study #3 the component had the shape of a simple ring/washer and was thus slightly further away from the sensing array (i.e., the component inner diameter was roughly the same as the outer diameter of the component in FIG. 4).

Findings of this case study included:
1. Sensors with refined configurations of dexamethasone-eluting components exhibited a positive, strong, and statistically significant oxygen signal effects at the sensor array. FIG. 1D illustrates oxygen levels measured by sensor electrodes without dexamethasone-eluting components 614 and those with dexamethasone-eluting components 416 in this study.
2. Sensors of the second type, all with dexamethasone-eluting components, implanted at three different depths exhibited: (1) reduced oxygen signals when implanted directly on the muscle and (2) typical oxygen signals when implanted directly on the fascia and in the subcutaneous fat. FIG. 1E illustrates oxygen levels measured by sensor electrodes with dexamethasone-eluting components, implanted on muscle 418, fascia 420, and subcutaneous fat 422.

In this study, (1) the refined dexamethasone-eluting component demonstrated enhanced oxygen delivery to the sensor array, consistent with previous studies, and (2) the sensors of the second type exhibited typical oxygen levels when implanted in subcutaneous fat or on the fascia, but experienced reduced oxygen levels when implanted directly on the muscle.

### Elution Model and Assumptions

As a basis for constructing its exemplary embodiments of substance delivery components for use in implantable devices, the Assignee hereof modeled the delivery of such substances (e.g., dexamethasone) within the tissue of the implant recipient based on several observations and assumptions. Specifically:

1) a first observation is that the mitigation of FBR occurs where the drug delivery component(s) touches bodily tissue; i.e., the anti-inflammatory effect of dexamethasone compounds is highly localized to where the dexamethasone is released.

2) Similarly, there is little if any “crossover effect” between areas exposed to the target substance (such as dexamethasone) and those which are not. Stated differently, exposure of one localized area of the tissue to the substance does not produce a significant broader, larger-area effect on inflammation or FBR. Hence, the introduction of anti-inflammatory substances such as dexamethasone may be very pointedly targeted at discrete areas, and its effects will remain highly localized.

3) Further, for several reasons including user safety, it is desired to keep the amount of free or systemic dexamethasone in the user's bloodstream low, but immediately available only to where the sensor and its active elements are. Corticosteriods may have adverse systemic and other effects in large enough quantities, and hence it is desirable to avoid large-scale introduction of these substances into the user's bloodstream. As such, the ability to use lower dosing of such substances is highly desirable.

4) Additionally, it has been observed by the Assignee hereof that in general, inflammatory response due to implantation of e.g., an analyte sensor tends to occur more rapidly during the initial phase of the implantation; i.e., shortly after implantation. The inflammatory response may tend to subside or be moderated thereafter. As such, there is in some applications a “window” during which the effect of any anti-inflammatory such as dexamethasone may have maximal efficacy, after which its efficacy is somewhat reduced (by virtue of the inflammatory response being reduced). Hence, exemplary delivery profiles described herein may make use of this observation, such that an initial “burst” or surge of substance (e.g., dexamethasone) delivery is used, followed by a more moderate delivery over time.

5) In order for anti-inflammatory substances such as dexamethasone to be effective in reducing inflammation in long-term implantation applications, a small amount of the drug generally must be slowly eluted over at least portions of the life of the implant.

Using the foregoing observations and assumptions, the Assignee hereof has applied a mathematical model to the elution of the target substance from one or more implanted sensor components over time. In one exemplary approach, the release was assumed to follows what is known as the Higuchi model. See “Strategies to Modify the Drug Release from Pharmaceutical Systems”, Woodhead Publishing, 2015, incorporated herein by reference in its entirety. It will be appreciated that while the Higuchi model is used as the basis of the subsequent discussion, the present disclosure is in no way so limited, and in fact other models may be used alone or in combination with Higuchi modeling, such as for different target substances, different phases of the delivery/implantation, or for other factors.

In the exemplary embodiment, the Assignee hereof has confirmed that the Higuchi model well describes the elution of drugs from this and other matrix systems (such as transdermal patches), and can be presented in a simplified manner as follows:

Q=A\sqrt{t} (Eqn. 1)

where Q is the cumulative drug release, t is time, and A is the release constant. Based on this equation it follows that the instantaneous drug release at any point in time is given by:

$$\frac{dQ}{dt} = \frac{0.5 A}{\sqrt{t}} \qquad ( Eqn . 2 )$$

Assignee has discovered that an “A” value of approximately 50-100 keeps the average daily release in the desired target range. With “A”=100, the day 60 (highest) release rate is 6.5 μg/day. With “A”=50, the day 420 (lowest) release rate is 1.2 μg/day. Looking ahead to potentially longer implant durations, with “A”=50 the release rate after two years of implantation is approximately 0.9 μg/day.

TABLE 1
Modeled DXA Release for One-year Implant

| A | 50 | 60 | 70 | 75 | 80 | 90 | 100 |
|---|---|---|---|---|---|---|---|
| Average Daily Release, | 1.8 | 2.1 | 2.5 | 2.7 | 2.8 | 3.2 | 3.6 |
| Days 60-420 (μg/day) | | | | | | | |
| Total Release, | 1.0 | 1.2 | 1.4 | 1.5 | 1.6 | 1.8 | 2.0 |
| Days 0-420 (mg) | | | | | | | |

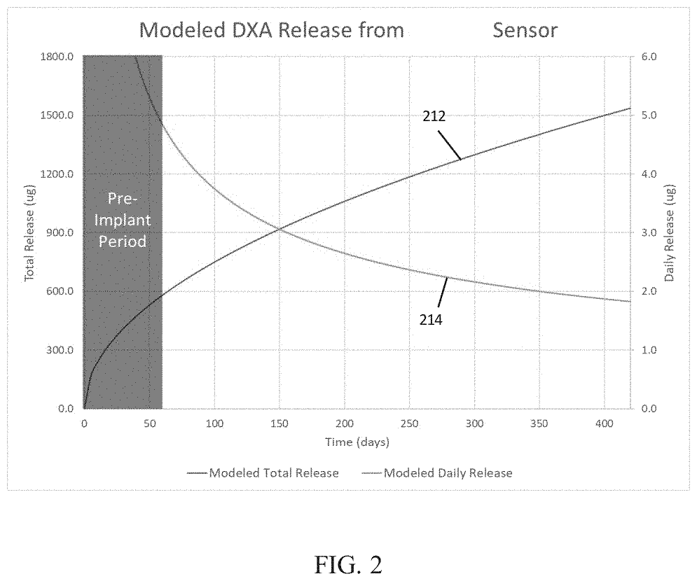

Table 1 shows an exemplary modeled total release through one year of implantation, for the exemplary substance DXA. The starting quantity of DXA on the sensor is configured to exceed these amounts in order to ensure the release profile occurs as designed. See also FIG. 2 for total and daily release profiles (212, 214) over time. Although the data presented above refers to DXA, it is contemplated that other dexamethasone compounds (e.g., DSP) would follow the Higuchi model with a similar release profile albeit with a different daily release amount, for example. However, as previously noted, the present disclosure also contemplates use of different models for different substances where appropriate.

The exemplary elution model results in an initial burst of release of the target substance, which slows and steadies over time. As previously noted, FBR and other bodily responses are more likely to occur and/or with higher intensity when a foreign object is initially implanted (i.e., FBR is also a “burst” response of sorts). Hence, the elution rate and profile according to this model advantageously corresponds to the rate of bodily responses.

In some embodiments disclosed herein, elution is modeled to begin during manufacturing, prior to sensor implantation. Notably, unlike traditional devices which are packaged “dry” (i.e., not immersed in any fluid environment when packaged), one exemplary embodiment of the disclosed implantable sensor device may be packaged in a saline solution until implantation, which alters the initial release of dexamethasone relative to a dry packaging scenario. This approach provides several advantages in the context of the present disclosure. Specifically, it allows for sufficient drug loading for long-term operation without exposing a patient to an excessive “burst” resulting in systemic exposure (e.g., per Case Study 1 discussed supra). The initial burst which would otherwise occur is mitigated by virtue of dissolution of some of the “loaded” substance within the saline.

Moreover, the foregoing approach allows for creation of an approximately linear release or zero order (constant) release rate without necessitating more complex component design. Stated differently, more complex designs to produce the desired substantially linear release profile are obviated by mitigating the initial non-linear “burst.”

It is further noted that, as e.g., DXA has a comparatively low water/saline solubility, elution into the saline storage solution occurs to only a certain extent. This gives the advantages discussed supra while still allowing of extended storage/shelf life (i.e., the DXA will not be completely or even substantially depleted prior to implantation such that the availability of the substance over the desired long term of the implantation is adversely affected).

With a manufacturing process that consists of at least two months (60 days) from the start of elution until implant, and using data from in vitro release testing, a daily release of between approximately 2 and 5 μg DXA in one exemplary embodiment is expected over the life of the implant (average of approximately 3 μg/day). These exemplary elution rates are within range of the elution rates seen in the above-referenced predicate devices. At these elution rates, it can be reasonably expected that patient plasma DXA levels to be below the 50 pg/mL LOQ. The full modeled release profile is shown in FIG. 2, which illustrates total release 212 of DXA from the implantable sensor device accumulating prior to implantation with, daily release 214 of DXA from the implantable sensor starting at a rate of approximately 4.8 μg per day upon implantation and gradually reducing to below 2.0 μg/day over the period of implantation. In other embodiments, DSP, other dexamethasone compounds, and/or other eluting anti-inflammatory drugs may be used similarly.

As previously noted, from a patient safety perspective, minimizing potential exposure to drug is preferred. At the maximum release profile (A=100), approximately 2 mg of drug is needed for a one-year sensor implant. Based on development testing by the Assignee hereof, delivery components with the nominal thickness (0.0045″) contain approximately 3.5 mg of drug. At a maximum allowed component thickness of 0.006″, the expected loading is approximately 4.7 mg. Advantageously, this “worst-case” exposure is still at the low end for a daily therapeutic dose, thereby further underscoring the inherent safety of the device when so configured; i.e., by controlling elution to low daily rates over a long period of time, the entire reservoir of the substance can be small and hence pose no systemic threat.

### Exemplary Implantable Sensor and Sensing Region

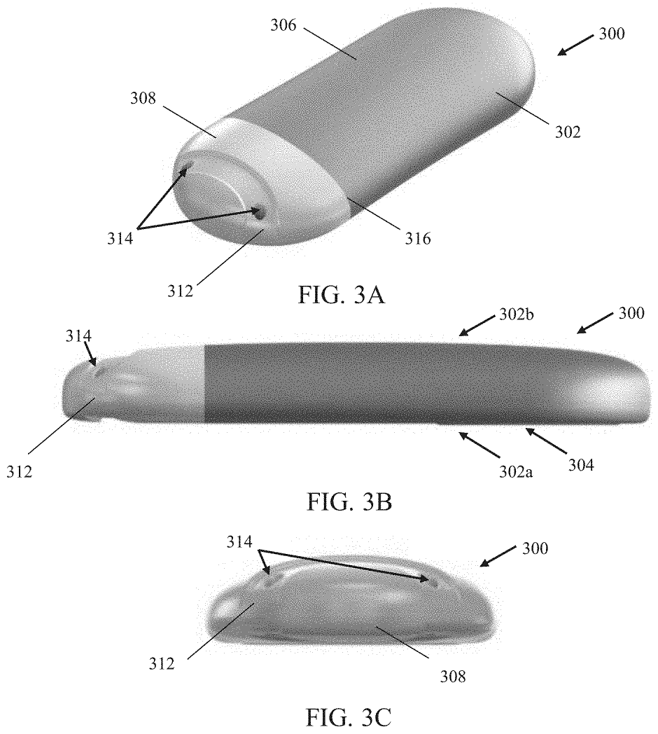

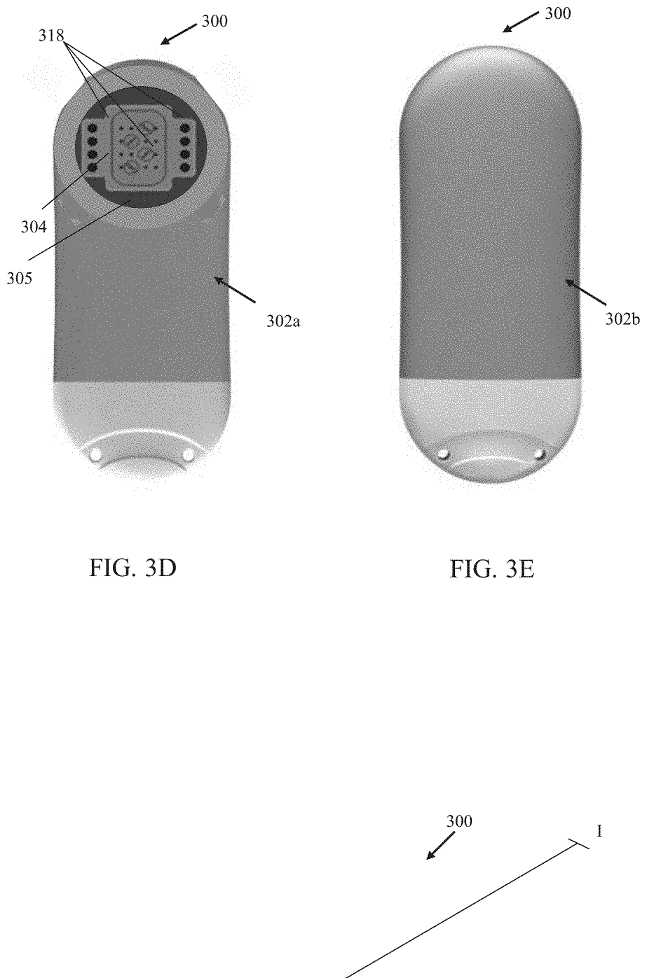

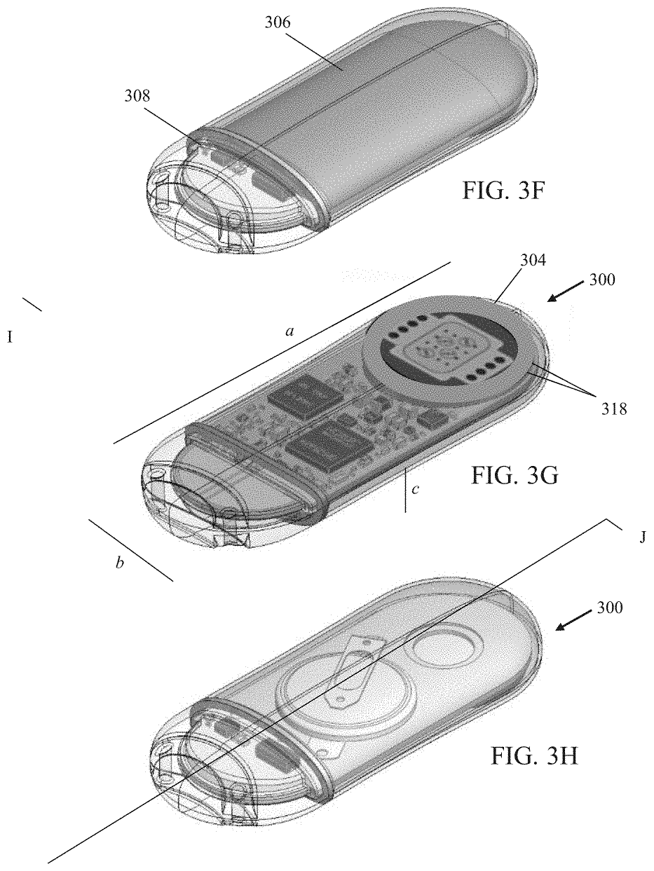

Referring now to FIGS. 3A-4, one exemplary embodiment of a sensor apparatus useful with various aspects of the present disclosure is shown and described.

As shown in FIGS. 3A-3H, the exemplary sensor apparatus 300 comprises a somewhat planar (and somewhat ovaloid) housing structure 302 having rounded opposing ends and substantially smooth and/or curved surfaces. A sensing region 304 is disposed on one side of the housing structure 302 (i.e., on a top face 302a, which opposes a bottom face 302b). As discussed in greater detail with respect to FIGS. 3A-7 herein, the exemplary configuration of the sensing region includes one or more substance (e.g., drug, such as dexamethasone) carrying components 305 which are configured to deliver the substance during at least part of the implantation lifetime of the device 300.

The exemplary substantially planar shape of the housing 302 provides mechanical stability for the sensor apparatus 300 after implantation, thereby helping to preserve the orientation of the apparatus 300, mitigating any tissue response induced by movement of the apparatus while implanted, as well as increasing comfort of the host/user. As described in greater detail subsequently herein, in some embodiments it is desirable to utilize the foregoing planar shape in conjunction with some level of body response (e.g., encapsulation) relative to areas of the sensor apparatus 300 which are not the sensing region 304, such as for mechanical stability and avoidance of movement of the implant during its implanted lifetime. This approach also reduces the quantity of active substance such as dexamethasone which must be used to effect control of the body response; i.e., only the sensor sensing region 304 is “controlled” in some variants since other areas of the implant 300 have no active sensing regions or other functions which would be impeded by encapsulation due to FBR.

Moreover, the rounded ends of the sensor apparatus 300 advantageously facilitate surgical insertion through one or more incisions formed within the subject as referenced elsewhere herein, and further give the apparatus 300 a less detectable and more comfortable profile after implantation due to, inter alia, the absence of sharp edges or corners. Further, the rounded opposing ends and substantially smooth and/or curved surfaces of the housing (as well as substantially eliminated seams between the housing components after assembly) aid in mitigation and/or limitation (overall) of foreign body response (FBR) after implantation of the sensor, thereby enabling long-term implantation and increasing accuracy of the sensor. Notwithstanding, the present disclosure contemplates sensor apparatus of shapes and/or sizes other than that of the exemplary apparatus 300.

The housing 302 of the illustrated embodiment includes two separable portions, a main body 306 and a cap 308. The main body 306 is comprised of a biocompatible metallic material, such as titanium or aluminum, which provides structural rigidity of the sensor housing and protects internal components of the sensor (as depicted in e.g., FIGS. 3F-3H). The cap 308 is comprised of a signal transmissive material (i.e., one which has at least some permeability to electromagnetic radiation such as radio frequency signals in the desired band(s) of interest), such as ceramic or a polymer, which permits an antennae 310 to transmit and receive signals. The cap is joined to the main body at a sealed seam 316 (e.g., a welded seam). Advantageously, the use of a single seam (and welding thereof) helps maintain the fluid-tight (and air-tight) integrity of the apparatus after assembly and implantation.

As can be seen in FIGS. 3A-3E, the cap 308 includes a grasping feature 312 and a plurality (two in this instance) of through-holes or anchor apparatus 314 disposed within an area of the grasping feature. In the depicted embodiment, the grasping feature comprises a curved and circumferential depression with the cap, which is utilized for manipulation of the sensor apparatus during manufacturing and/or implantation and can be “grasped” by hand, or with an appropriate tool (e.g., forceps). Anchor apparatus 314 provide the surgeon with the opportunity to anchor the apparatus to the anatomy of the living subject (via receipt of sutures (dissolvable or otherwise), tissue ingrowth structures, and/or the like therein), so as to frustrate translation and/or rotation of the sensor apparatus 300 within the subject immediately after implantation but before any tissue response (e.g., FBR) of the subject has a chance to immobilize (such as via interlock with the sensing region of the apparatus). See e.g., U.S. patent application Ser. Nos. 14/982,346 and 15/197,104, for additional details, considerations, and configurations regarding the aforementioned anchor apparatus and sensor implantation.

In alternate embodiments, the grasping feature and/or the anchoring apparatus may have different configurations. For example, the grasping feature may have ridged configuration or a textured surface to increase grip, and/or the anchoring apparatus may be raised above the surface of the end cap as e.g., eyelets (and not penetrate through a portion of its thickness as in the embodiment of FIG. 3D). In other examples, the grasping feature and/or the anchoring apparatus may be eliminated from the cap, giving the cap a substantially smooth surface. Further, anchoring features may additionally or alternatively be included in a different region of the housing, such as within or projecting outwardly from the main body. Furthermore, the anchoring features may have a gel-like substance disposed therein (e.g., a silicone or silicone-based compound) in order to discourage tissue growth into or within the anchoring features, thereby easing a subsequent explant of the sensor and limiting damage to surrounding tissues during the explant procedure.

As can be seen in e.g., FIGS. 3D and 3G, the sensor apparatus further includes a plurality of individual sensor elements 318 with their active surfaces disposed substantially within the sensing region 304 on the top face 302a of the apparatus housing, and immediately proximate the substance containing component 305. In the exemplary embodiment (e.g., an oxygen-based glucose sensor), the multiple sensing elements 306 are disposed in groups on the sensor face, one element of each group being an active or “primary” sensor with enzyme matrix, and the others being reference or “secondary” (oxygen) sensors associated with the proximate primary sensor (which are unassociated with any enzyme matrix). Exemplary implementations of the sensing elements and their supporting circuitry and components are described in, inter alia, U.S. Pat. No. 7,248,912, U.S. patent application Ser. Nos. 15/170,571; 15/359,406; and Ser. No. 16/233,536, each previously incorporated herein.

In the illustrated embodiment of FIGS. 3D and 3G and 4, the substance delivery component 305 effectively circumscribes two rectangular regions associated with the primary glucose sensor elements and auxiliary sensor elements, respectively.

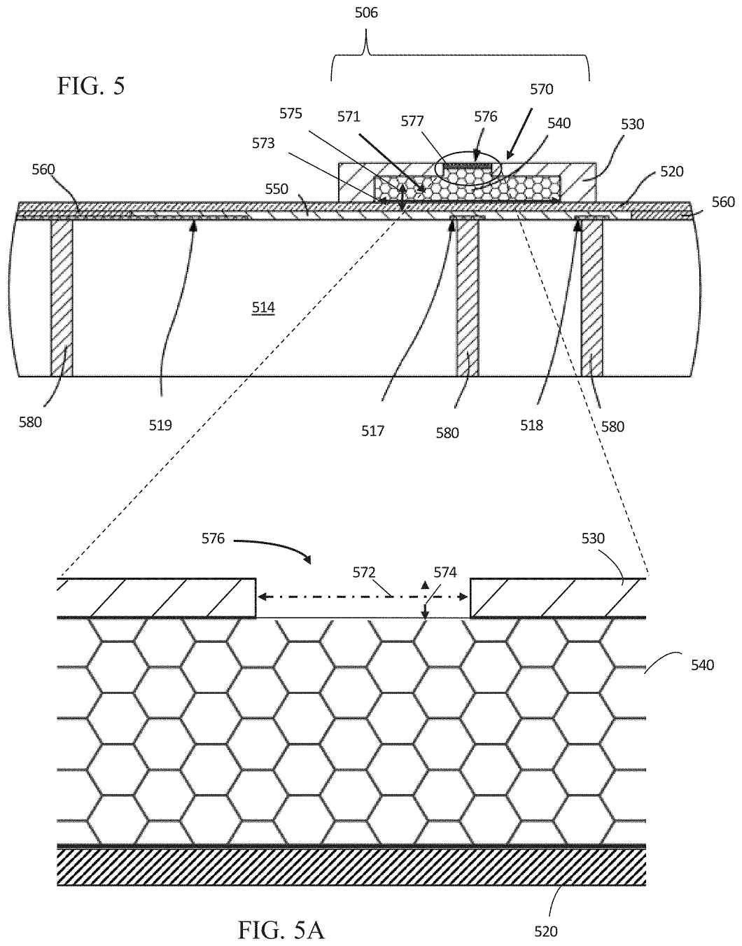

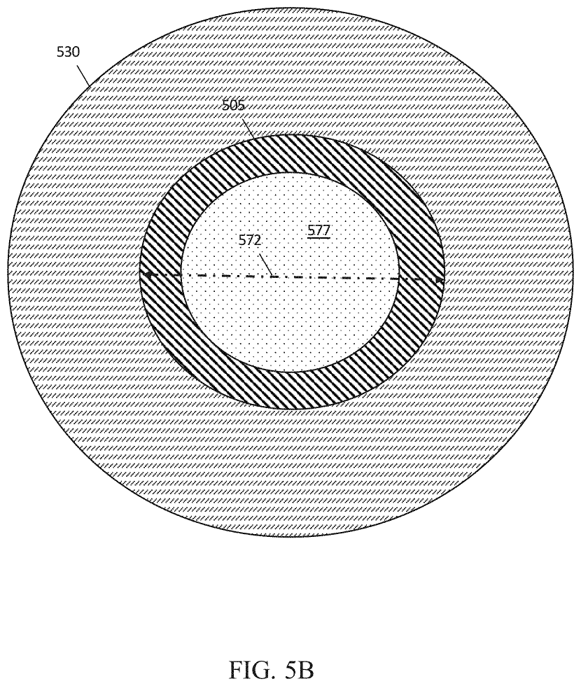

Specifically, in reference to FIG. 4, the “vertical” rectangle 402 contains the silicone rubber outer device membrane (e.g., element 506 in the exemplary embodiment of the detector shown in FIG. 5 discussed infra). The four (4) larger circles 407 (shown in an exemplary “zig-zag” pattern) are enzyme cavities (e.g., corresponding to element 571 in the exemplary configuration of FIG. 5). Below this visible top level reside the working electrodes (not shown in FIG. 4), responsible for measuring the glucose-modulated and background oxygen signals. The reference electrodes 409, also within this rectangle 402, are part of the voltage control circuit for the working electrodes.

The horizontal rectangle 411 allows space for the counter electrodes 413 (discussed further infra), which sink the current of the working electrodes. It is noted that these electrodes 413 off-gas during operation, and as such remain uncovered in the exemplary configuration shown. An ionically conductive layer (not shown) connects each counter electrode to its respective working and reference electrodes.

The configuration used in the embodiment of FIG. 4 advantageously enables at least portions of the delivery component to be spatially proximate to the sensor elements, as close as possible so that the inflammation-controlling effects of the substance carried in the component 305 has maximal efficacy for the sensor elements.

It will be appreciated that the configuration of the component 305 as shown in FIG. 4 is but one example; other shapes and/or disposition of the various components may be used consistent with the present disclosure. For instance, the generally circular outer peripheral shape may be changed to be e.g., square, hexagonal, oval, or some other shape depending on factors such as the shape of the underlying implant 300. As another alternative, the outer peripheral shape may be contoured consistent with the inner circumscribed shapes (e.g., the two rectangles discusses above) such that an effectively constant width (in a direction radial from the center of the sensor region) is achieved. Additionally, the interior circumscribed shape(s) may be altered, such as where a single circular or other filed shape containing each of the sensor elements discussed above) may be used. Myriad other possibilities exist, as will be recognized by those of ordinary skill when given the present disclosure.

It will further be appreciated that the type and operation of the sensor apparatus may vary; i.e., other types of sensor elements/sensor apparatus, configurations, and signal processing techniques thereof may be used consistent with the various aspects of the present disclosure, including, for example, signal processing techniques based on various combinations of signals from individual elements in the otherwise spatially-defined sensing elements pairs. Moreover, other exemplary embodiments of the sensor apparatus described herein may include any of: (i) multiple detector elements which can have respective “staggered” ranges/rates of detection operating in parallel, and/or (ii) multiple detector elements, optionally having respective “staggered” ranges/rates of detection, that are selectively switched on/off in response to, e.g., the analyte concentration reaching a prescribed upper or lower threshold, or a certain sensor group or type being optimal under specific conditions, such as those described in the foregoing U.S. patent application Ser. No. 15/170,571, previously incorporated herein. The present disclosure further contemplates that such thresholds or bounds, and/or sensor groups: (i) can be selected independent of one another; and/or (ii) can be selected dynamically while the apparatus 300 is implanted. For example, in one scenario, operational detector elements are continuously or periodically monitored to confirm accuracy, and/or detect any degradation of performance (e.g., due to equipment degradation, progressive FBR affecting that detector element, etc.). If degradation is detected, e.g., affecting say a lower limit of analyte concentration that can be detected, a particular detector element can be “turned off” or have its signals removed from data calculations, such that handoff to another element capable of more accurately monitoring concentrations in that range or under those specific physiological conditions occurs. Note that these thresholds or bounds are to be distinguished from those associated with the user interface (UI) described subsequently herein, the latter being independent of the data source/capability/configuration associated with the sensor detector elements.

Additionally or alternatively, embodiments of the presently described sensor apparatus can include heterogeneous sensor elements of first and second types, such as e.g., oxygen-based sensor elements and hydrogen peroxide-based sensor elements, each configured for detection of blood glucose. In one such implementation, the implantable sensor apparatus includes both oxygen-based sensor elements and hydrogen peroxide-based sensor elements, one of the element types acting in a confirmatory or calibration capacity to in effect “second check” and adjust (as necessary) the other sensor elements, thereby ostensibly extending the interval between other confirmatory processes utilized with the device (e.g., fingersticking), and/or the implant-to-explant interval, hence improving user experience with the device and quality of life.

It will be appreciated that in the case of such heterogeneous sensor types (some of which may invoke more or less inflammatory response intrinsically due to their principles of operation, such as a peroxide-based detector which characteristically causes more inflammatory response due to exposure of tissue to peroxides which are irritating in nature), differential substance delivery components and/or profiles may be used in conjunction therewith. For instance, such peroxide-based detectors may warrant a higher initial dose and/or elution profile over time of the anti-inflammatory agent as compared to the exemplary oxygen-based sensors described above. To implement this, various embodiments of the sensor apparatus can include multiple different substance delivery components, such as where those immediately proximate to one type of detector element are configured for one delivery profile and/or eluted substance, while those proximate another type of detector element are configured for a second, different delivery profile/substance.

In one exemplary configuration, the first and second analyte detector element types are used in parallel, and one detector type acts as a reference for the other detector type (e.g., the hydrogen peroxide-based glucose detector element is a reference for the oxygen-based glucose detector element, the oxygen-based glucose detector element is a reference for the hydrogen peroxide-based glucose detector element, etc.). In another example, the first and second analyte detector element types are used in parallel and measurements or readings from both detector element types are used to generate or derive a composite measurement (e.g., via a weighted average). In yet another example, the first and second analyte detector types can be alternately and/or selectively employed depending on specific implantation, use, and/or physiological conditions.

In one variant, the various heterogeneous detector elements (e.g., detector elements of the first glucose detector type and the second glucose detector type, and/or detector elements of various configurations for either sensor type according to specified ranges of sensitivity and/or rates of detection) can be selectively switched on/off (even while the sensor apparatus is in vivo), so as to, e.g., accommodate “on the fly” changes to blood glucose concentration or other physiological changes occurring within the host, or to maintain efficacy of the detector elements within a known or desirable range of accuracy or sensitivity. One type of detector can also be prioritized over another, or swapped out, such as e.g., where the performance of one detector type has eroded over time (due to e.g., FBR associated with that particular detector), or loss of some other desirable attribute or performance aspect. It is also recognized that different detector elements of the same type may in fact experience different localized inflammatory responses for any number of reasons, and as such two otherwise identical detector elements (which are also effectively identical in terms of proximity to the substance delivery component(s)) may none-the-less have a differential operating characteristic over time, and as such may be selectively included within populations or sets of detectors which can be operated earlier or later within the implantation time period. For instance, detector elements indicating a more rapid initial decay of pO2 levels may be selectively operated earlier within the implantation period, whereas those indicating less inflammatory-related effect may be preserved or operated later within the period.

Specific examples of the foregoing heterogeneous detector element sensors are shown and described in the U.S. patent application Ser. No. 15/359,406, previously incorporated herein. It will be appreciated that an on-board processor of the sensor and its associated logic can be configured to perform the foregoing selective use (e.g., turning on/off) of sensor elements.

Returning to FIGS. 3D and 3G, in addition to being configured to have (either homogenous or heterogeneous) sensing elements disposed therein, the sensing region 304 or portions peripheral thereto may be configured to facilitate some degree of “interlock” of the surrounding tissue (and any subsequent tissue response generated by the host) so as to ensure direct and sustained contact between the sensing region 304 and the blood vessels of the surrounding tissue during the entire term of implantation (as well as advantageously maintaining contact between the sensing region 304 and the same tissue; i.e., without significant relative motion between the two). See e.g., U.S. patent application Ser. No. 15/197,104 filed Jun. 29, 2016 and entitled “Bio-adaptable Implantable Sensor Apparatus and Methods,” now U.S. Pat. No. 10,638,962, previously incorporated herein, for additional details and considerations regarding utilization of the user's foreign body response (FBR) to at least partially generate interlock between aspects of the sensor apparatus and the host tissue.

It will be appreciated that the relatively smaller dimensions of the sensor apparatus (as compared to many conventional implant dimensions)—on the order of 53 mm in length (dimension “a” on FIG. 3G) by 22 mm in width (dimension “b” on FIG. 3G) by 7 mm in height (dimension “c” on FIG. 3G)—may reduce the extent of injury (e.g., reduced size of incision, reduced tissue disturbance/removal, etc.) and/or the surface area available for blood/tissue and sensor material (i.e., non-active portions of the sensor) interaction, which may in turn reduce intensity and duration of the host wound healing response, and increase longevity of the implant due to greater signal stability over time, especially in conjunction with the reduced inflammatory response in the sensing region 304 afforded by the substance delivery component(s) 305. It is also envisaged that as circuit integration is increased, and component sizes (e.g., Lithium or other batteries) decrease, and further improvements are made, the sensor may increasingly be appreciably miniaturized, thereby further leveraging this factor; i.e., the ratio of inflammation-reduced or controlled tissue to non-controlled tissue such as that in contact with the remainder of the implanted apparatus 300 will increase, and the overall amount of “inflamed” tissue will decrease, thereby resulting in reduced inflammatory effect on the operation of the sensor.

### Exemplary Drug-Eluting Device Component

Referring now to FIG. 4, one exemplary embodiment of the substance delivery component 305 of the implantable sensor device 300 is shown and described in detail.

It is evident from the previously discussed predicate studies and Assignee's own findings that continual release of a small dose of a target substance (e.g., dexamethasone acetate) into the tissue near the sensing area may be utilized to reduce fibrous tissue encapsulation of the device without any systemic effects. As such, the drug-eluting component 105 of the implantable device is capable of releasing a safe and effective amount of drug over the lifetime of the sensor, as benchmarked against predicate devices. In various exemplary embodiments, the exemplary drug-eluting component(s) 105 is/are used with the implantable device as shown in FIGS. 3A-3H, e.g., in conjunction with the sensing region 304.

Ideally, the eluting component 305 is placed as close as possible to the active sensor area 304 of the device, and to elute an average of approximately 2-5 μg/day of anti-inflammatory substance (e.g., DXA in one exemplary embodiment) during the implanted lifetime. In order to avoid detectable systemic levels of DXA, daily release is in some embodiments configured to be below approximately 15 ug, which keeps DXA levels well below those associated with cortisol suppression.

In one exemplary embodiment of the drug-eluting component 305 as shown in FIG. 4, the drug-eluting component being incorporated in the implant device consists of micronized dexamethasone compounds (e.g., dexamethasone acetate (DXA) or dexamethasone sodium phosphate (DSP)) immobilized within a substrate. In various embodiments, a substrate made of implant-grade silicone rubber (SR) is used. In other variants, other materials may be used as a matrix, such as polyester urethane, polyether urethane, nylon, resins, thermosets, etc.

In the illustrated embodiment, a sheet of the cured silicone 305 may be adhered or otherwise disposed or incorporated onto an appropriate surface area of the sensor 300, such as the area proximate or surrounding the sensing elements 304, as shown in FIG. 4. Myriad shapes of the silicone sheet are possible in various implementations. As previously noted, the use of a sheet that is circumscribed by two rotated rectangular shapes corresponding to primary and auxiliary sensors is illustrated in this example, although other configurations will be appreciated by those of ordinary skill given this disclosure.

The placement of the silicone sheet proximate the sensing elements may also facilitate any necessary (e.g., indirect) measurement of locally present dexamethasone or the effects thereof, such as via determination of the thickness of the fibrous encapsulation of at least a portion of the sensor face. Such determinations may be made using any number of different techniques, such as e.g., external imaging modalities (e.g., X-ray, CAT, ultrasound or similar), internal sensing modalities (e.g., impedance, resistance, strain, piezoelectricity), or indirectly via observation of certain detector element parameters such as pO2 over time (e.g., via capture and periodic or opportunistic wireless transmission of data off-implant to an external receiver). Post-explant examination and measurement may also be performed; i.e., when the surgeon removes the extant implant, the level of encapsulation can be quantified. This determination can be correlated to e.g., the level of dexamethasone which has been delivered over time (and/or its efficacy). Such measurements may factor into a determination of subsequent actions to be taken, whether with respect to the current patient on the current iteration of implantation, or other patients and/or iterations. For instance, a less-than-expected level of encapsulation for a given individual may be used to guide whether the level of anti-inflammatory substances (including dexamethasone) should be adjusted in subsequent implants; e.g., the DXA loading of the next sensor implanted may utilize less DXA (even when considering the reduced sensitivity/encapsulation expected on subsequent implantations as discussed in detail subsequently herein). In effect, this particular patient may have less inflammatory response than originally modeled, and hence DXA loading on one or more subsequent implants may be reduced.

Additionally, the data for this patient may be used during the term of the current implantation (for the same patient) to adjust operation of the implant, or other associated parameters such as implantation duration. As a simple example, less-than-expected encapsulation of the sensor region may militate in favor of longer implantation lifetime for the currently implanted device (consistent with other considerations described elsewhere herein). Additionally, the operation of the detectors themselves of the current implant may be adjusted “on the fly” using e.g., wireless data communication with the implant as described in one or more of Assignees' patents/application previously incorporated by reference herein. For example, BLE protocol wireless commands may be inserted into the implant causing it to perform one or more of: (i) selective electrical/logical substitution or replacement of one detector element (or one type of detector element, such as peroxide-based vs. oxygen-based) of the implant with another; (ii) removal of one or more detector elements which may not be operating as expected from contribution to the calculated output data; (iii) changes in weighting of individual detector elements relative to others, such as where the determined level of encapsulation in one region of the sensor is asymmetric with that of another, such that certain detector elements may suffer less “direct” or overhead encapsulation and as such be better suited for longer-term operation of the implant, and/or (iv) release of additional substance (e.g., DXA) where the implant is configured to controllably elute or dispense additional DXA (e.g., via an additional onboard reservoir which may be controlled after implantation).

Yet further, the data relating to determination of encapsulation (e.g., thickness) for the given patient may be used in conjunction with similar data from one or more other patients so as to better statistically model the inflammatory response of patients as a whole to certain configurations of the implant. It may be for instance that the data from a given patent indicates that they are somewhat of a data “outlier” in one regard or another, and hence a different type or configuration of detector/implant (or protocol for use of the detector/implant) is appropriate. These results may also be correlated to e.g., genetic, environmental, physiologic (e.g., height, weight, age, sex, location, ethnicity, implantation history, use of certain pharmaceuticals such as confounding acetaminophen, and the like) or other factors such as via machine learning (ML) or other approaches to identify statistically significant factors in inflammatory response for individual patients or the patient population as a whole) as the relate to inflammatory response and/or specific eluted substances such as DXA, and utilized with respect to the present patient and/or others, whether in the current implantation iteration or subsequent one(s). In one exemplary implementation, the DXA and silicone (SR) are mixed in dry powder form prior to molding and curing of the SR, resulting in a generally uniform suspension of DXA in sheet having a diameter of 0.5″ and thickness of 0.0045″ at a concentration of 20% w/w. Further, a target surface area of approximately 0.2 in² was determined for the sensor component to achieve a nominal/desired release profile over the long term (e.g., one year). For the exemplary nominal release profile, a coefficient value of “A”=75 according to Eqn. 2 discussed above with respect to the so-called Higuchi model is selected, while holding other parameters constant (drug loading %, materials, etc.).

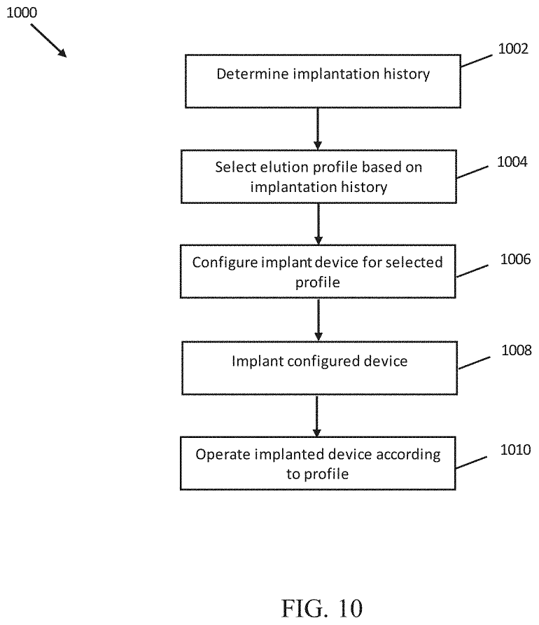

Those having ordinary skill in the art will recognize, given this disclosure, that other amounts or concentrations of the anti-inflammatory substances, or thicknesses (thinner or thicker than 0.0045 inches) and/or dimensions (diameter, width, or length) and surface area(s) of the silicone sheet(s) may be selected to “tune” the elution mechanism to accommodate one or more of: (i) the size of the implantable device, (ii) a desired elution profile over time, (iii) the surface area available proximate to the sensing elements, and/or the sensing element configurations, (iv) the level that a patient is susceptible or immunocompromised (less dexamethasone may be necessary), and (v) prior implantation history of the patient (there is lesser need for dexamethasone after the first implant; i.e., on second and subsequent implants, a lesser amount or no dexamethasone may be necessary as discussed with respect to FIG. 10 herein).

Moreover while dexamethasone/DXA are generally used as an exemplary substance, it will be appreciated that other anti-inflammatory substances, such as the aforementioned DSP, or other corticosteroids (e.g., prednisone) or yet other types of agents with desirable properties, may be used for similar or complementary effect.

Based on the dimensions of the silicone sheet in the exemplary implementation, the amount of DXA loaded onto the sensor may be approximately 3.5 mg. However, other amounts of DXA (or DSP, etc.) may be loaded based on current or estimated level of FBR, thickness of the silicone, size of the implanted device, history, etc.

Advantageously, it has been further shown through Assignee's studies that manufacturing of the exemplary drug-eluting component poses no issues for shelf life of the implant apparatus 300. Release profile and stability, as well as drug loading remaining after the initial (pre-implantation) release profile, have remained stable. It has been shown thereby that the exemplary dexamethasone-loaded silicone sheet (e.g., patch) can be stored at room temperature for extended periods and remains stable.

Further, in some other embodiments of the substance delivery component 305, a selective mix of different types of dexamethasone may be employed. Rather than loading the silicone sheet with DXA only, a mix of DXA and dexamethasone sodium phosphate (DSP) may achieve different elution rates and/or profiles as is desired. More specifically, in one exemplary approach, dexamethasone and any cohorts thereof (e.g., various mixtures of different types of dexamethasone, and/or other substances with desirable properties) are added to silicone rubber matrix as a powder dispersed throughout rubber matrix before the rubber is cured. In one variant, a homogeneous (i.e., evenly distributed) mixture is used to enable consistent elution from the rubber. For instance, in one implementation, a 90:10 mix of DXA and DSP would increase water solubility as compared to DXA only, and thereby provide a somewhat different elution rate/profile from the silicone sheet, particularly during the initial burst that occurs upon implantation (discussed below). A quicker/slower release profile may also be suitable for implants that have a lower/longer shelf life, or which due to be explanted and replaced in the patient sooner/later.

Various spatial distributions of DXA (or other substance) concentration may also be used consistent with the present disclosure. In one embodiment, non-uniform “domains” having higher or lower concentrations of dexamethasone compounds (e.g., DXA) may be created within the silicone, such as by curing the rubber before the dexamethasone powder has dispersed, or applying other techniques which differentially distribute the target substance within the matrix, followed by curing. In one variant, multiple stages of mixing may be performed to create the domains, where in one stage, the mixing is uniform, and in another stage, the dexamethasone is not allowed to distribute uniformly. In another embodiment, multiple silicone sheets or (vertical) layers having different concentrations of dexamethasone compounds may be utilized, such that the concentration varies a function of depth into the component 305. For instance, multiple sheets (e.g., two sheets each having less than 3.5 mg of DXA loaded thereon in different concentrations/loadings, such that the substance loading of the second (obstructed) sheet takes longer to migrate to the tissue through the first overlaid layer of material).

In yet a further variant, the distribution of the target substance may be controlled laterally; e.g., areas closer to the sensor elements of the implant may have a different concentration than those further away, whether according to a discrete (e.g., step) or continuous function.

In yet other implementations, multiple silicone sheets (“patches”) may be used with the sensor elements, with at least one having DXA and at least one having dexamethasone sodium phosphate mixed with the respective sheets.

In other implementations, the delivery or elution component has a geometric cross-sectional shape, such as circular, oval, square, trapezoidal, etc., such as where it is not a flat sheet or patch as described above. Such approaches may be used for example to adjust to contours of the host implant/device 300, or in order to increase surface area exposed to the tissue (e.g., a crowned or bowed profile exposes more surface area and hence potentially greater contact area with the host's tissue in that region).

In a further implementation, the component is formed as two or more discrete distributed components, such as across or proximate to an active region of an implantable sensor face. For instance, the patch 305 may in fact be comprised of two or more “mini-patches” disposed at different locations on/proximate to the sensor face.

### Exemplary Detector Elements

In some embodiments, the exemplary drug-eluting component(s) is/are used with other extant components of the implantable device 300, including within components of one or more of the analyte detector elements themselves. For instance, as shown in FIGS. 5 and 5A, the substance delivery components may be used in conjunction with an enzymatic oxygen-based detector element such as those described in, e.g., co-owned U.S. patent application Ser. Nos. 15/368,436 and 14/982,346, and 15/170,571, each incorporated supra. Such a detector element includes an enzymatic matrix contained within a membrane shell structure (made of e.g., silicone rubber) and forms a small opening or aperture, e.g., in a spout formation, through which hydrophilic enzymatic material is disposed for diffusionally accepting solutes of interest (e.g., glucose, oxygen). In some embodiments, the shape and dimension of the spout region aids in controlling the rate of entry of the solute of interest into the enzymatic material.

To further illustrate, as shown in FIG. 5, an exemplary individual (primary) detector element 506 of the type noted above is shown associated with detector substrate 514 (e.g., a ceramic substrate), and generally comprises a plurality of membranes and/or layers, including e.g., the insulating layer 560, and electrolyte layer 550, an enzymatic gel matrix of the type described above 540, an inner membrane 520, an exterior membrane shell 530, and a non-enzymatic membrane 577. Such membranes and layers are associated with the structure of individual detector elements, although certain membrane layers can be disposed in a continuous fashion across the entire detector array surface or portions thereof that include multiple detectors, such as for economies of scale (e.g., when multiple detectors are fabricated simultaneously), or for maintaining consistency between the individual detector elements by virtue of making their constituent components as identical as possible.

The exemplary sensor apparatus includes both primary (enzymatic) and secondary (non-enzymatic) detector elements; while FIG. 5 illustrates one embodiment of the former, the latter is in the exemplary embodiment generally similar in structure, with the exception that the enzymatic matrix 540 is replaced with a non-enzymatic matrix or other structure; i.e., in the case of a glucose-sensitive primary detector, it does not include glucose oxidase and catalase (as described in U.S. patent application Ser. No. 15/170,571 incorporated above). It will be appreciated, however, that the construction of primary and secondary detector elements need not be substantially similar, and in fact may differ significantly in construction so long as the desired performance attributes are maintained.

As shown in FIGS. 5 and 5A, a single spout region 570 of the detector element 506 forms a small opening or aperture 576 through the membrane shell 530 to constrain the available surface area of hydrophilic enzymatic material 540 exposed for diffusionally accepting the solute of interest (e.g., glucose) from solution. Alternatively, it is contemplated that one or more spout regions (and/or apertures within a spout region) can exist per detector element.

The exemplary spout 570 is formed out of the hydrophobic material of the membrane shell 530 without bonded enzymes (e.g., silicone rubber) and advantageously includes a non-enzymatic outer layer or membrane 577 to, inter alia, prevent direct contact of the immobilized enzymes in the enzymatic material 540 with the surrounding tissue, thereby eliminating and/or reducing antibody response (e.g., FBR), encapsulation, and/or other deleterious factors. In exemplary embodiments, the non-enzymatic membrane 577 is further constructed (i.e., with a substantially planar crosslinked biocompatible matrix possessing pores substantially smaller than those required to accommodate blood vessel ingrowth, but large enough to accommodate diffusion of solutes of interest) so as to frustrate or mitigate blood vessel formation therein.

Hence, as can be appreciated by those of ordinary skill given this disclosure, one or more of the aforementioned membrane structures 530, 577 may be loaded, coated, and/or or impregnated with one or more substances of interest for the control of inflammatory response. In one such approach, each of the silicone shell membranes 530 of a given implant apparatus is loaded (similar to the sheet or patch previously described) with DXA and/or other substances if desired so as to achieve a desired elution profile after implantation. As with the sheet/patch 305, these shells 530 may have their total loading and elution modeled so as to either act as the sole substance delivery component(s) within the sensor 300, or alternatively work in concert with other components (such as the patch 305) to create a desired overall profile, including one that contains two or more temporal portions controlled by the contributions of different ones of the delivery components (e.g., the patch 305 directed to a shorter-term burst of DXA, while the shells 570 are configured to deliver a longer term, more moderated dose).

In some embodiments of the disclosed sensor elements, the aforementioned (e.g., albumin) membrane 577 itself may include dexamethasone (e.g., DXA) and/or other substances, while isolating enzymatic materials from the tissue. This configuration advantageously allows both passage of oxygen into the enzyme matric of the detectors via the membrane 577, which is enhanced by virtue of the presence of the dexamethasone (i.e., the tissue immediately adjacent to the outer membrane 577 is less inflamed, and therefore produces less encapsulation which results in greater blood/analyte flow available to the detector.

Notably, in some embodiments, the membrane 577 may be configured with a more uniform distribution of DXA or the like (at least within one or more strata of the membrane or the membrane as a whole), or alternatively with differentiated concentrations, such as via one or more DXA “domains” within the membrane. For instance, an outer periphery or ring 505 of the membrane 577 may be loaded with DXA, while the interior (radially) regions are DXA free or at lower concentration, so as to avoid any effects of the presence of the DXA/other substances with the passage of the desired analytes through the membrane. See FIG. 5B. Moreover, the silicone shell membrane 530 may also be loaded with the same or different substance(s) as the ring 505, such as where one component (e.g., the ring) 505) is loaded one way and to one concentration so as to (primarily) address one operational or temporal region, whereas the membrane is loaded differently so as to (primarily) address another.

In some embodiments, further controlled release of dexamethasone (or other e.g., anti-inflammatory substances) and configuration of elution rates may be desirable. For example, additional DXA (or DSP) may be loaded and “delay released” after a period of time determined by e.g., the amount initially loaded and delivered. Such substances to be delayed-released may be stored inside or on the component with the initial substances, or another component to the device. For instance, if less than 3.5 mg of DXA initially loaded is substantially released after a certain period of time (estimated or based on local amount of substances measured by the device), a secondary (and/or tertiary, etc.) silicone sheet, membrane, or other structure having modified-release properties may be employed at that time to release additional DXA. On the other hand, the implant may include means of controlling the release of substances. For instance, in some embodiments, additional amounts of acetate may be included during the formation/molding of the silicone sheet or membrane so as to capture free dexamethasone and thereby cause a slower release into the body. In some embodiments, additional amounts of other dexamethasone compounds such as DSP may be used to increase the elution rate into the body from certain components, while others are used for later portions of the implantation as referenced above.

For instance, in some applications, it may also be desired to have a more consistent release profile; i.e., the elution rate may be controlled to have a smaller variance than between 2 and 5 μg (approximately) daily release as shown in FIG. 2. To this end, in some embodiments, secondary amounts of drug (e.g., DXA) may be delayed-released after a certain number of days (e.g., 150 days may be selected based on model-estimated daily release amount of DXA falling to 3.0 μg) or based on local amount of DXA detected by the device (e.g., DXA falling to 3.0 μg or other selected threshold amount). In some embodiments, extended-released or other forms of controlled-released dosages are possible. Additionally, in other embodiments, slower elution may be achieved by choosing one or more material qualities or parameters such as e.g., a higher density or thickness of the matrix into which the drug is impregnated, and vice versa.

In some embodiments, yet other existing components of the implantable device (e.g., non-sensor components rather than those of the detector as shown in FIGS. 5-5B) may be reconfigured to be a source of dexamethasone.

In addition to design considerations including material selection as described above, in order to further stabilize the elution of anti-inflammatory drugs (e.g., DXA or DSP) from the sensor component, each lot of SR components 305, 530, 577 may subjected to one or more quality control tests with respect to, e.g., geometric dimensions of the silicone sheet or the sensor area of the implant device, total drug content, drug content uniformity, in vitro drug release kinetics, in vivo drug release kinetics, and drug identity testing.

Thus, in the various exemplary implementations, the substance delivery components are to varying degrees ideally disposed as close as possible to the active sensor elements 304 of the implant, so as to have maximal efficacy of the substance (i.e., at suppressing inflammatory response in a localized fashion) while minimizing the overall drug loading and hence systemic distribution thereof. Moreover, different amounts, levels of controlled release, or mixtures of dexamethasone and or other compounds may be determined based on individual patient considerations such as tolerance to corticosteroids, history of implantation, characterizations of prior inflammatory responses at the implantation site (e.g., the patient is unusually susceptible or prone to FBR, or unusually not susceptible/prone), by customizing the shapes, positions, and/or drug loadings of the silicone patches or other components.

A further advantage of having localized areas that are “protected” from FBR is that the remaining portions of the implant 300 may be used to facilitate immobilization of the implant in a cavity or pocket within the tissue and be secured within the patient via encapsulation, as alluded to in co-owned U.S. patent application Ser. No. 14/982,346 filed Dec. 29, 2015, entitled “Implantable Sensor Apparatus and Methods”, and issued as U.S. Pat. No. 10,660,550 on May 26, 2020. These unprotected or exposed portions of the implant (and thereby the degree of encapsulation) may be controlled based on usage of the patches 305 and/or other components 530, 577.

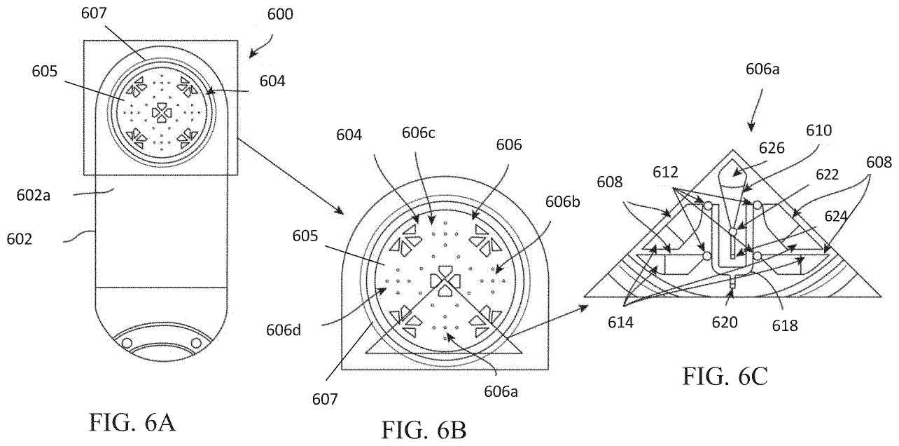

Referring now to FIGS. 6A-6C, another exemplary embodiment of a fully implantable sensor apparatus 600 having a sensor face 604 with drug-eluting capability is depicted. Aspects of this embodiment are further described in co-owned U.S. patent application Ser. No. 16/453,794 filed Jun. 26, 2019 entitled “Apparatus and Methods for Analyte Sensor Spatial Mismatch Correction”, which claims priority to co-owned and co-pending U.S. Provisional Patent Application No. 62/690,745 filed on Jun. 27, 2018 and entitled “Apparatus and Methods for Analyte Sensor Spatial Mismatch Correction,” each incorporated herein by reference in its entirety.

As can be seen in FIG. 6A, the exemplary sensor apparatus 600 comprises a housing 602 having a sensor face 604 (sensing region) disposed on a top surface 602a thereof, generally similar to that previously described with respect to FIGS. 1A-1H herein. The sensor face 604 includes four groups of sensing elements radially arranged thereon, one of which, sensing element group 606a, is highlighted in FIG. 6B and shown in greater detail in FIG. 6C. The sensor face 604 also includes a thin membrane sheet 605 (of thickness similar to those previously described) which is loaded with a desired level of anti-inflammatory such as DXA or a mixture of substances as described elsewhere herein. A ring 607 which surrounds the sensing face 604 may also be utilized (whether alone or with the sheet membrane 605) so as to achieve the desired drug-eluting profile(s). In some variants, the ring 607 and sheet membrane 605 are made of silicone rubber as previously described, with the sheet being punctuated with openings or apertures for the unimpeded passage of analyte species of interest to the detector elements. To that end, the exemplary sheet circumscribes the various features of the detectors/sensor face closely so as to deliver the substance as close to the detection points as possible, yet does not impede the detector elements' operation.

As can be seen in FIG. 6C, the group of sensing elements 606a includes multiple background species sensing elements 608 (e.g., four background oxygen elements) associated with and proximate to a single analyte-modulated sensing element 610 (e.g., one glucose-modulated oxygen element) for an enhanced spatial relationship of background species and analyte-modulated signals. In alternate embodiments, the sensor face may in include additional or fewer groups of sensors, and/or additional or fewer background (oxygen) elements associated with each analyte-modulated (glucose) element. Additionally, in the present embodiment, each of the sensor element groups has a configuration which is substantially similar to other sensor groups; however, in alternate embodiments, the sensor elements within each group may have a different configuration/arrangement than that of the other groups (e.g., group 606b having a different configuration than group 606a). Similarly, it will be appreciated that the sheet membrane 605 may also be differentially constructed if desired, such as via four “quadrants” (not shown) which each may have similar or different properties (i.e., drug loading, elution profile, or other) than the others.

Returning to the embodiment of FIG. 6C, the four background oxygen elements 608 each include a background oxygen (BO) working electrode 612 associated with a BO counter electrode 614. In the exemplary configuration of FIG. 6C, the BO counter electrodes 614 are substantially disposed at opposing lateral sides (proximate to an outer perimeter) of the sensing element group. The orientation of the BO counter electrodes toward the outer perimeter of the sensing element group enables a closer arrangement of the BO working electrodes to the glucose-modulated sensing element. Specifically, the BO working electrodes 612 are evenly-spaced and arranged around a glucose-modulated (GM) working electrode 622 in a substantially square-shaped and clustered configuration.

It will further be appreciated that the sheet membrane 605 used on the sensor face 604 may further be configured as a non-enzymatic membrane configured to pass analytes of interest therethrough while also having some drug loading, similar to the albumin-based membrane 577 previously described with respect to FIGS. 5-5B. For example, the membrane may be uniform (i.e., no punctations or apertures) and both (i) allow species of interest to migrate towards the detector elements, and (ii) contain sufficient substance loading to achieve the desired elution profile (whether alone or in combination with the ring 607 if used).

### Methods

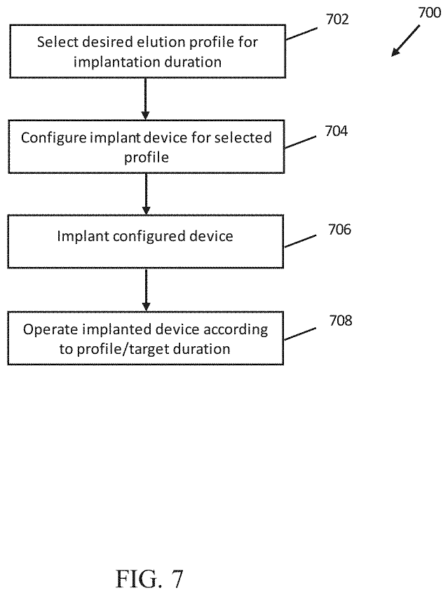

FIGS. 7-10 illustrate exemplary embodiments of various methods of using and configuring the sensor implant apparatus as previously described. In some embodiments, the implantable device used may be that shown in FIGS. 3A-3H. In other embodiments, the implantable device may be that shown in FIG. 5-5A or 5B. In further embodiments, the implantable device may be that shown in FIGS. 6A-6C. In yet other embodiments, other types implantable devices may be used, as described in, e.g., co-owned U.S. patent application Ser. Nos. 15/368,436, or 14/982,346 or yet others, incorporated supra.

Moreover, to the degree applicable, at least portions of the following methods may be implemented within computer programs or applications resident on the implanted device itself, one or more external computerized platforms such as a smartphone, tablet, wrist-worn or similar external receiver or accessory, or even cloud-based systems. For example, changes in pO2 values over time may be detected and evaluated using on-device logic of the implant, and communicated wirelessly to an application operative to execute on a user's smartphone.

FIG. 7 illustrates an exemplary method for operating an implantable apparatus of the type described previously herein. As shown in FIG. 7, the method 700 includes selection of the desired elution profile(s) for a target implantation duration. For instance, if the implant is intended or total implant period of 18 months, the elution profile(s) are selected accordingly so as to optimize the inflammatory response mitigation and control during that period, such as by front-loading the elution of the delivered substance during the first few weeks of implantation, and then progressing to a more stable eluation rate thereafter. As previously noted, the effect of any elution towards the tail end of the implant period may be minimal or negligible, and hence for shorter implant periods, the total drug loading may in some embodiments be reduced relative to a longer implantation period, with the drug effectively exhausted as the close of the period approaches. Alternatively, if it is desired to provide some “design margin” for the implantation period (such as where the ability to explant at a certain time is unknown), the profile and/or loading may be adjusted so as to stretch out the period.

Per step 704 of the method, the implant is configured for the selected profile/period. For instance, the loading of the patch 305 and/or other components (e.g., membranes 577, 530 or 605, 607) if used is adjusted to achieve the profile. As such, the present disclosure contemplates fabrication of a number of different components which are each adapted to particular profiles/parameters, such that the device may be specifically adjusted for a given user's case (i.e., individual-specific configuration). This may even be accomplished by the implanting physician, such as be selecting from one of a plurality of differently preconfigured devices, or ordering a customized device e.g., via Internet portal, such as before the implantation procedure commences (e.g., JIT or “just in time” delivery of the implant shortly before scheduled implantation) so as to maximize its efficacy.

Per step 706, the configured device is implanted within the patient/host, and per step 708 is subsequently operated for the implantation lifetime according to the target profile.

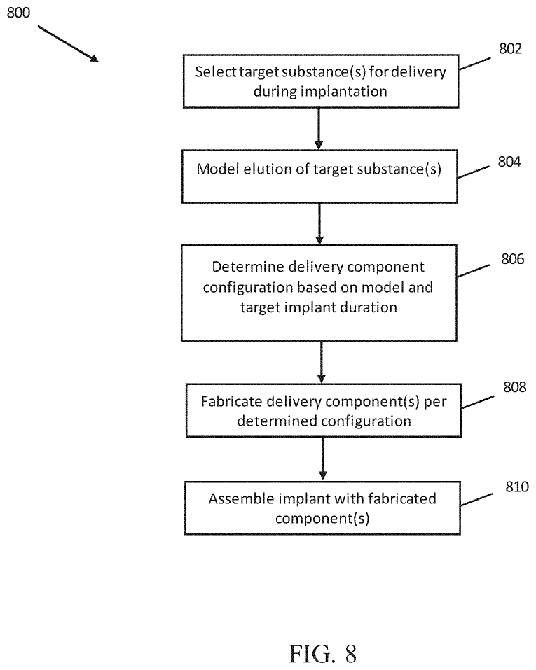

FIG. 8 illustrates one embodiment of a method of fabricating the implant apparatus described herein. Specifically, as shown, the method 800 first includes selecting one or more target substances for delivery via the implant. For instance, a single substance such as DXA may be selected, or alternatively a mixture of dexamethsoane variants may be used (whetehr together within one delivery component, or discretely in two or more delivery components). As discussed above, DXA is a corticosteroid capable of inhibiting inflammation, thus improving availability of oxygen and glucose to the sensor in addition to reducing fibrous encapsulation. In other embodiments, other dexamethasone compounds, other corticosteroids, or other forms of anti-inflammatory drug may be chosen alternately or in addition to DXA, such as dexamethasone sodium phosphate (DSP). Hence, in another embodiment, (DSP) is chosen as the anti-inflammatory substance. In some variants, a prescribed mixture of DXA and DSP (or other suitable corticosteroids) is identified, as discussed elsewhere herein. However, in the following exemplary methods, references to DXA will be made with the understanding that DSP and/or one or more other anti-inflammatory substances may be substituted or combined with DXA.

The foregoing substance selection may also be with a particular patient or host in mind, or more generically based on e.g., a desired implantation period.

Per step 804, the eluation mechanism(s) for delivery of the target substance(s) is/are modeled. As previously discussed, Higuchi modeling may be used for some substances; yet, the present disclosure contemplates that two or more different models may be appropriate in some circumstances (such as where the selected substances differ in terms of physical characteristics, transport mechanisms within the tissue, transport mechanisms within the delivery components into which they are loaded, etc.

Per step 806, based on the selected substances to be delivered and their delivery/elution models, the configuration of the component(s) used for delivery is determined, It is noted that steps 804 and 806 may be combined or iterative with one another, in that the selected components (e.g., use of silicone rubber versus another polymer) for the component may impact the delivery modeling). Steps 804 and 806, when completed, converge on a configuration of delivery components (including degree and mechanisms of loading of the target substance(s)) which may be used in the apparatus 300 to be implanted within the target host.

Per step 808, once the delivery component configuration(s) is/are defined, the components can be manufactured according to the design definition or specifications.

Lastly, per step 810, the implant apparatus is assembled with the fabricated component(s) such that it is ready for implantation in the target host.

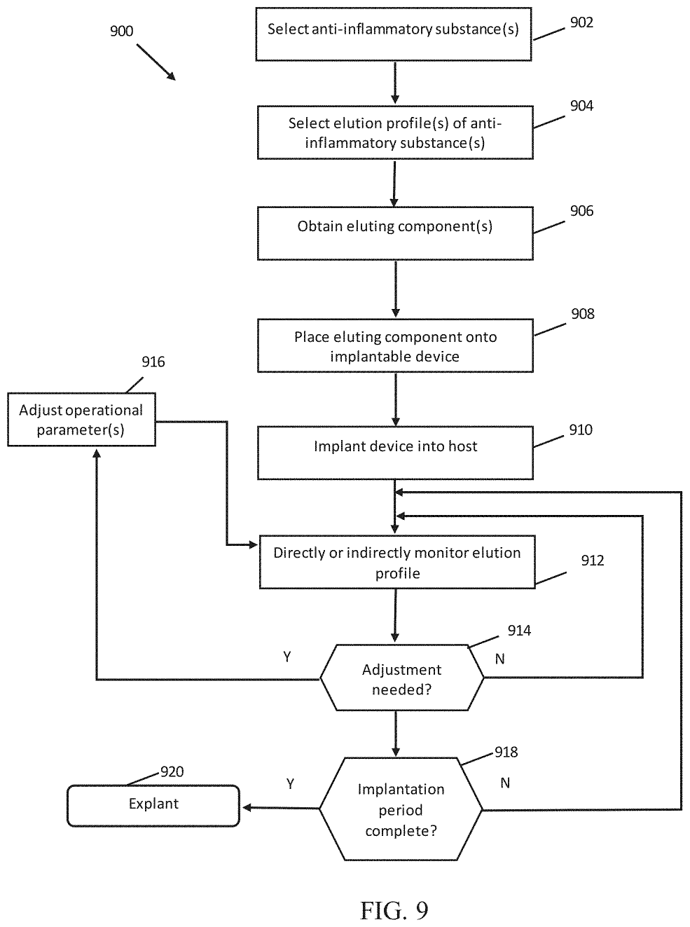

FIG. 9 shows an exemplary method for mitigating and controlling anti-inflammatory bodily reactions caused by an implantable device during implanted operation thereof.

At step 902, one or more anti-inflammatory substance is/are identified. In one exemplary embodiment, dexamethasone acetate (DXA) is chosen.

At step 904, an elution profile of the selected anti-inflammatory substance is selected. In one exemplary embodiment, selecting the elution profile includes selecting a desired elution rate for at least one portion of the implantation period. In one such embodiment, an average of 3 μg of DXA released per day over the lifetime of the sensor is selected. Alternatively, or in addition, a range of elution rate is selected. In one exemplary embodiment, an elution rate of 2-5 μg/day over the lifetime of the sensor is selected. That is, it may be desired to release the anti-inflammatory substance at an initial rate of approximately 5 μg/day, and near the end of service of the implanted sensor, it may be desired to release at approximately 2 μg/day. As noted, the release of the substance slows over time (see Eqn. 2). Elution rates, times, and characteristics may be adjusted according to the desired application and factors as described elsewhere herein (e.g., a patient with a prior implantation does not require as much FBR mitigation).

At step 906, an eluting component (such as patch component 305 and/or membranes 577, 530) is manufactured and obtained for an implantable device according to the selected anti-inflammatory substance and elution rate. In one exemplary embodiment, DXA may be immobilized onto a matrix or substrate such as a sheet of implant-grade silicone rubber (SR) to produce a drug-eluting component (e.g., a DXA-eluting component). The silicone sheet may have various shapes and dimensions appropriate for the implantable device and the sensor elements (discussed further below).

In one exemplary embodiment, the DXA and SR are mixed prior to molding and curing of the SR, resulting in a uniform suspension of DXA in 0.0045″ thick sheet at a concentration of 20% w/w. The silicone sheet in the exemplary embodiment takes on a somewhat ring shape as shown in FIG. 2.

In some embodiments, other concentrations of DXA, or thicknesses (thinner or thicker than 0.0045 inches) and/or dimensions (diameter, width, or length) of the silicone sheet may be selected based on the selected elution rate for the DXA and/or the size of the implantable device. For example, if a higher elution rate is desired, a higher concentration of DXA may be mixed into the mold, and vice versa. In some embodiments, dimensions or size of the component may be based on the selected elution rate. More specifically, if a higher or lower elution rate is desired, the diameter, length, width, and/or thickness of the component may be sized to be larger or smaller, respectively. In some other embodiments, as discussed above, the component may have controlled-release properties so as to allow faster or slower elution.

At step 908, placement of the substance-eluting component on the implantable device is selected. In one exemplary embodiment, the component 305 once it is formed in step 906, is roughly ring-shaped (see FIG. 2) and placed around a sensor portion, disposed proximate sensing elements. However, myriad other placements of incorporation according to available space (external or internal to the implant), shape of sensor area, etc. may be possible in other embodiments. For example, in one variant, multiple strips of the component may be placed proximate the sensing elements. In another variant, multiple substance-eluting components may be placed on the implantable device so as to enable different levels of exposure to the tissue in different regions, or for other purposes.

At step 910, the implantable device (with sensor and substance-eluting component installed) is placed into a host via surgical implantation. In one exemplary embodiment, the device is placed inside a patient, avoiding direct contact with muscle tissue, and rather placed near subcutaneous fat or fascia.

At step 912, the operation of the implant is monitored. In one embodiment, the implanted device includes short-range communication interfaces such as Bluetooth Low Energy or 433 MHz systems. This monitoring may include direct measurements of the eluted substance(s) such as via a sensor of the implant configured to detect it, and/or “indirect” monitoring of the elutions, such as by the effects on pO2 or other parameters which may be affected by the presence or absence of the delivered substance(s). For example if the pO2 profile of the device sensor elements drops more rapidly over time than expected, it can be surmised that the eluting or delivery components are either not functioning as intended, or the eluted substance if largely ineffective at inflammatory control in this particular application. Conversely, if an expected profile of pO2 values over time is consistent with expected norms such as previously collected data for this or other patients, then the elution mechanisms can be considered to be operating as expected with the desired efficacy.

In one variant, the indirect monitoring of the elution rate is estimated based on an elution model (such as that described previously herein). Given the time of manufacturing and/or the time of implantation, an approximate rate may be determined and correlated to an expected pO2 level. In one implementation thereof, the sensing element(s) may be employed to check in at certain times (e.g., every day or other set time period and/or on predetermined days after manufacture or implantation) to confirm that the readings are in line with the estimation of the elution rate.

Per step 914, if the monitoring at step 912 indicates that some adjustment of the elution configuration or other parameters of the implantation (e.g., the target duration needs to be shortened), thet at step 916 such adjustment is made. In one variant, this includes adjusting the implantation period as noted above. Note that adjustments may work in both directions; if e.g., the monitored pO2 levels show less-than-expected decline over time (indicative of perhaps less inflammatory response), then the implantion period may be extended subject to other considerations such as battery life, patient desires, regulatory considerations, etc.

Other possible adjustments may include changes to operating parameters or algorithms within the implant detector logic itself. For example, mechanisms by which “impeded” or poorly performing individual detector elements are removed from use of have their relative weightings changed (see e.g., the mechanisms described in the foregoing co-owned applications/patents incorporated by reference) may be made more or less sensitive such that detector accuracy overall is increased, ostensibly compensating for greater-than-expected pO2 degradation over time. As another option, calibration intervals and/or criteria may be changed, such as requiring the user to conduct more frequent confirmatory fingersticks or the like.

Data generated by the monitoring and and adjustments made may also be logged or stored into the implant's device memory for e.g., transmission off-implant to an external device via wireless interface, or even extraction from the device at time of explant.

Lastly, per step 918, the operation of the implant is continued for the duration of the implantation period (whether the original period, or as adjusted per step 916), at which time the implant is explanted per step 920.

FIG. 10 illustrates one embodiment of a method of utilizing the improved sensor apparatus disclosed herein for one or more successive implantations. Specifically, as shown in FIG. 10, the method 1000 includes first determining an implantation history for the target host/patient. For instance, the planned implantation may be the first which the patient has undergone, or it may be the second, or third, and so-forth. The assignee hereof has identified that during such subsequent implantations, especially those which use the same implantation site or “pocket” such as described in co-owned U.S. Pat. No. 10,660,550 incorporated herein, the level of FBR/inflammatory response may markedly decrease. In effect, the first implantation of a cycle “shocks” the body into a response which than later de-amplifies or insulates against similar response of the same magnitude. This may conceivably be explained by the formation of the fibrous encapsulation around the pocket; the body in effect may sense that it has already erected a protective barrier, and hence further responses can be less comprehensive. Regardless of the actual physiologic mechanism, this observation can be leveraged during e.g., use of implants such as those of FIG. 3A-4, 5-5B, or 6A-6C, so as to tailor the configuration of the implant for the particular iteration or place within the foregoing cycle. For instance, it may be desirable to reduce the total loading of a given substance such as DXA of a given implant so as to reduce systemic introduction. There may also be other as of yet unidentified benefits to reducing the loading of certain substances (whether corticosteroids or otherwise) of an implant which can be accommodated using the methodologies described herein.

Returning to the method 1000, per step 1004, an elution profile is selected based on the patient's implantation history. For instance, if the planned implantation is the second implantation for that subject, modeling and analysis or even heuristics (e.g., reduce by half for each subsequent implant) may be used as a basis of configuring the implant device (step 1006) for that individual (or group of individuals, if the response characteristics on a per-implantation basis can be generalized).

Per step 1008, the configured device is implanted, and per step 1010, the device is operated according to the profile. Note that the methodology (or portions thereof) of the method 800 of FIG. 8 can be combined with that of FIG. 10, such that e.g., after implantation, the effects of the eluted substance can be directly or indirectly monitored, and any necessary operational adjustments made. For instance, it may be that the particular patient shows and even more aggressive reduction in FBR/inflammatory response than expected, and hence the operational lifetime of the device can be extended subject to other prevailing considerations (e.g., battery life) as previously described.

It is further noted that at some point, it may be that no substance delivery or elution is required (e.g., on the fourth or fifth implant for that individual).

The foregoing methodology 1000 also underscores the general desirability of utilizing the same implantation site for a given patient multiple times. Specifically, once the “pocket” is formed (including its fibrous encapsulation), and assuming a similarly configured device/form factor with sensor face is used, each subsequent implanted device can simply re-use the same pocket and sensor face “window” formed over the prior implantation(s), and hence the physiology of the patient as whole is disrupted as minimally as possible, including the ability to reduce or even eliminate the use of the anti-inflammatory substances described herein..

It will be recognized that while certain embodiments of the present disclosure are described in terms of a specific sequence of steps of a method, these descriptions are only illustrative of the broader methods described herein, and may be modified as required by the particular application. Certain steps may be rendered unnecessary or optional under certain circumstances. Additionally, certain steps or functionality may be added to the disclosed embodiments, or the order of performance of two or more steps permuted. All such variations are considered to be encompassed within the disclosure and claimed herein.

While the above detailed description has shown, described, and pointed out novel features as applied to various embodiments, it will be understood that various omissions, substitutions, and changes in the form and details of the device or process illustrated may be made by those skilled in the art without departing from principles described herein. The foregoing description is of the best mode presently contemplated. This description is in no way meant to be limiting, but rather should be taken as illustrative of the general principles described herein. The scope of the disclosure should be determined with reference to the claims.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

What is claimed is:

1. A method of configuring an implantable device to mitigate at least one inflammatory bodily response, the method comprising:
identifying at least one anti-inflammatory substance for use with the implantable device;
determining a desired elution profile for the at least one anti-inflammatory substance; and
incorporating a substance-eluting component configured to provide the desired elution profile within the implantable device.

2. The method of claim 1, wherein the at least one anti-inflammatory substance comprises at least dexamethasone acetate (DXA) and dexamethasone sodium phosphate (DSP), the DXA and the DSP utilized in a prescribed proportion to achieve the desired elution profile.

3. The method of claim 1, wherein the substance-eluting component comprises a sensor membrane of an analyte detector of the device, the sensor membrane configured to pass at least one analyte to enable analyte detection by the analyte detector.

4. The method of claim 3, wherein the analyte detector of the device comprises an oxygen-based blood glucose detector, and the determining of the desired elution profile comprises determining a profile which is non-linear as a function of implantation time.

5. The method of claim 1, wherein:
the determining a desired elution profile for the at least one anti-inflammatory substance comprises:
determining a desired elution duration;
generating at least one model for elution of the at least one anti-inflammatory substance for at least the desired elution duration; and
determining at least one aspect of a configuration of the substance-eluting component, the determining of the at least one aspect based at least on the desired elution duration and the generated at least one model.

6. The method of claim 5, wherein the generating at least one model for elution of the at least one anti-inflammatory substance for at least the desired elution duration comprises using at least one machine learning (ML) algorithm to identify one or more factors in an inflammatory response as a function of the at least one anti-inflammatory substance.

7. The method of claim 5, wherein the using the at least one machine learning (ML) algorithm to identify one or more factors in an inflammatory response as a function of the at least one anti-inflammatory substance comprises application of the at least one ML algorithm to historical data relating to a plurality of living beings other than a living being within which the implantable device is to be implanted.

8. The method of claim 1, further comprising fabricating the substance-eluting component consistent at least with the determined at least one aspect.

9. The method of claim 1, wherein the determining the desired elution profile for the at least one anti-inflammatory substance comprises determining the desired elution profile based at least on data relating to one or more historical implantations of an implantable device of the same type as the implantable device.

10. The method of claim 9, wherein the determining the desired elution profile based at least on the data relating to one or more historical implantations of an implantable device of the same type as the implantable device comprises determining the desired elution profile based at least on data relating to one or more historical implantations within a same living being within which the implantable device is to be implanted.

11. The method of claim 1, wherein the historical implantations comprise implantations of implantable devices comprising apparatus to elute the least one anti-inflammatory substance.

12. An implantable sensor apparatus configured to mitigate undesired body response when implanted in a living being, the sensor apparatus comprising:
at least one analyte detector element;
a wireless interface configured to transact data wirelessly with a computerized device external to the living being;
digital processor apparatus in data communication with the wireless interface; and
at least one substance-eluting component disposed proximate the at least one analyte detector element, the at least one substance eluting component configured to elute at least one anti-inflammatory substance at least when the implantable sensor apparatus is implanted within the living being.

13. The implantable sensor apparatus of claim 12, wherein the at least one substance-eluting component is configured to elute the at least one anti-inflammatory substance according to an elution profile.

14. The implantable sensor apparatus of claim 13, wherein the elution profile is achieved based at least on (i) a physical configuration of the at least one substance-eluting component, and (ii) a loading at least one anti-inflammatory substance in the at least one substance-eluting component.

15. The implantable sensor apparatus of claim 14, wherein the physical configuration of the at least one substance-eluting component comprises a thickness of at least a portion of the at least one substance-eluting component.

16. The implantable sensor apparatus of claim 14, wherein the loading at least one anti-inflammatory substance in the at least one substance-eluting component comprises a loading as a function of a depth of the at least one substance-eluting component.

17. The implantable sensor apparatus of claim 12, wherein the at least one substance-eluting component is configured to elute at least some of the at least one anti-inflammatory substance before implantation.

18. The implantable sensor apparatus of claim 12, wherein the:
the at least one analyte detector element comprises an oxygen-based blood glucose detector element;
the at least one substance-eluting component comprises implant-grade silicone rubber (SR); and
the at least one anti-inflammatory substance comprises dexamethasone acetate (DXA), the DXA immobilized within at least a portion of the implant-grade SR.

19. The implantable sensor apparatus of claim 12, wherein the:
the at least one analyte detector element comprises an oxygen-based blood glucose detector element; and
the at least one anti-inflammatory substance comprises an acetate salt of dexamethasone acetate (DXA), the acetate salt of DXA comprising a solubility selected in order to achieve a desired eultion profile as a function of time.

20. A method of configuring an implantable sensor apparatus so as to extend its implantation lifetime, the implantable sensor apparatus comprising at least one anti-inflammatory substance-eluting component configured to interact with tissue of a living being upon implantation of the sensor apparatus therein, the method comprising:
determining a target implantation lifetime for the implantable sensor apparatus;
developing at least one model for elution of the at least one anti-inflammatory substance within the living being over the target implantation lifetime; and
based at least on the developed at least one model, configuring the substance-eluting component of the implantable sensor apparatus to elute the at least one anti-inflammatory substance at least during implantation according to one or more elution profiles, the one or more elution profiles configured to inhibit a foreign body response of the living being to the sensor apparatus so as to enable the sensor apparatus to operate within a prescribed level of accuracy for at least the target implantation lifetime.
